(19) United States
(12) Patent Application Publication
(10) Pub. No.: US 2024/0374563 A1
(43) Pub. Date: Nov. 14, 2024

(54) ANTICHOLINERGIC COMPOUNDS FOR USE IN TREATING NEUROMUSCULAR DISORDERS

(71) Applicant: UNIVERSITY OF MONTRÉAL, Montreal (CA)

(72) Inventors: Richard Robitaille, Montréal (CA); Danielle Arbour, Montréal (CA); Eric Martineau, Montréal (CA); Elsa Tremblay, Montréal (CA); Joanne Vallée, Montréal (CA)

(21) Appl. No.: 18/779,877

(22) Filed: Jul. 22, 2024

Related U.S. Application Data
(63) Continuation of application No. PCT/CA2023/050112, filed on Jan. 27, 2023.

(30) Foreign Application Priority Data
Jan. 28, 2022 (EP) 22154109.7

(51) Int. Cl. A61K 31/4025; A61P 21/00

(52) U.S. Cl. CPC A61K 31/4025; A61P 21/00

(57) ABSTRACT

The present invention relates to an anticholinergic compound such as Darifenicin for use in the treatment of a neuromuscular disorder (in particular amyotrophic lateral sclerosis or ALS) in a human subject, wherein the neuromuscular disorder is characterized by an impaired neuromuscular transmission and/or an denervation at the NMJ (neuromuscular junction) and the anticholinergic agent is administered after symptomatic onset.

### FIELD OF THE INVENTION

The present invention relates to an anticholinergic compound for use in the treatment of a neuromuscular disorder in a human subject.

### BACKGROUND

ALS is an adult-onset non-cell autonomous neuromuscular/neurodegenerative disorder which causes the progressive loss of upper and lower motor neurons (MN), leading to gradual paralysis and death in 2 to 5 years. Neuromuscular junction (NMJ) denervation is a hallmark of ALS [1] and is present in several disease models of ALS [2-6], even preceding the death of MN [1, 3].

The currently approved ALS treatment (Riluzole) benefits only 20% of ALS patients by extending their life for approximately three months. The effect of Riluzole on muscle function is very limited. Moreover, ALS is considered a genetically heterogenous disease likely representing several subgroups with differing underlying pathology. There is currently no cure available, nor will patient-tailored therapies likely be able to aid all ALS patients because of the different underlying disease mechanisms.

Therefore, there is still a need for a new therapy for ALS and other diseases that mimic ALS (‘ALS-like diseases’).

### LEGEND TO THE DRAWINGS

FIG. 1 Darifenacin target engagement: Darifenacin treatment dampens muscarinic hyperexcitability in SOD1 mice. A) Ca²⁺-responses induced in PSCs by local applications of muscarine from untreated SOD1 (left) and treated mice with two dosages of darifenacin (10 or 15 mg/kg). The mean amplitude for each animal is represented by a black circle and the average amplitude of Ca²⁺-responses of untreated SOD1 mice is represented by the light grey rectangle. The number of cells that responds to muscarine application (responsiveness) and the mean±SEM of the amplitude of Ca²⁺-responses elicited in PSCs following ATP application are indicated. B-C) Scatter plots illustrating the mean amplitude of Ca²⁺-responses elicited in PSCs by muscarine (B) or ATP local applications (C) in animals represented in A.

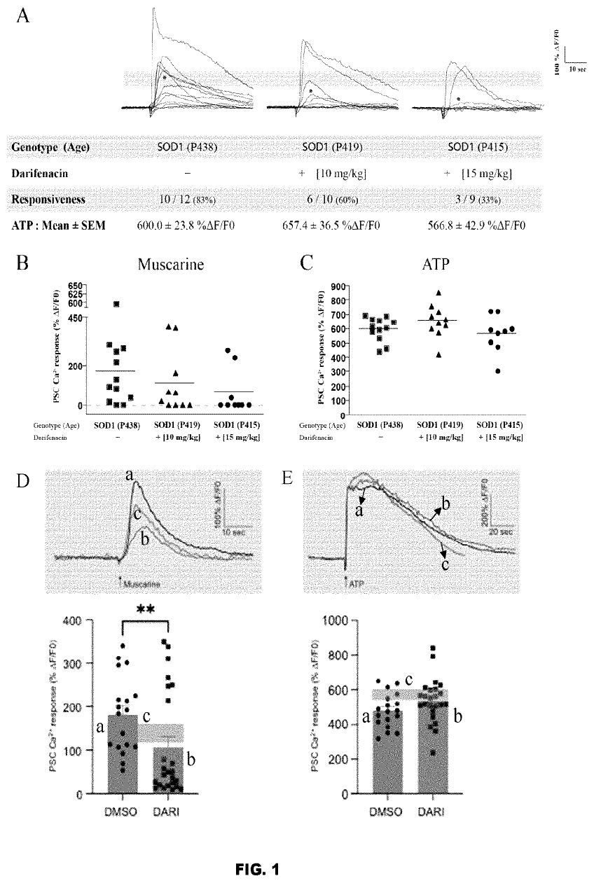

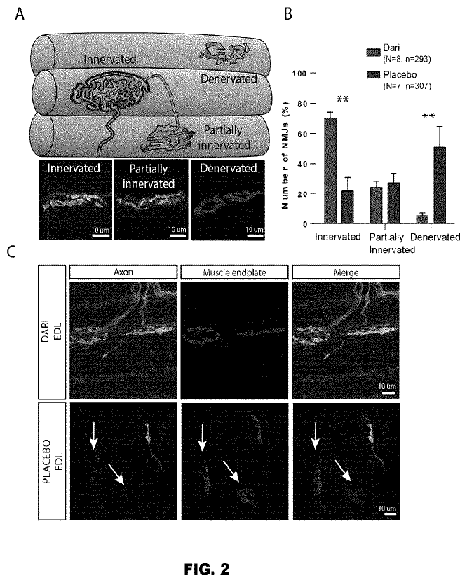

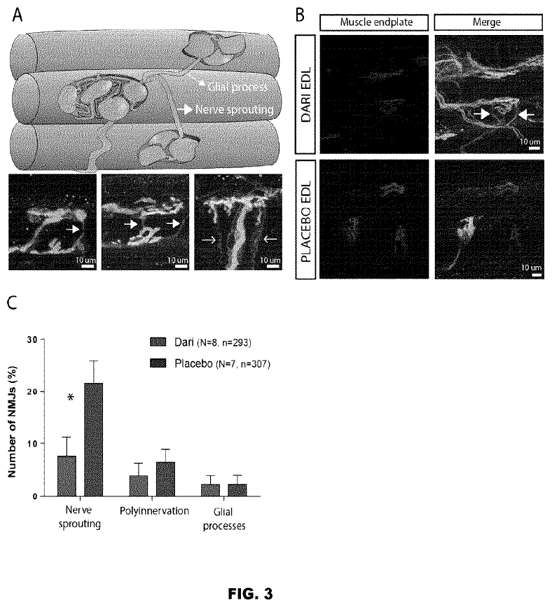

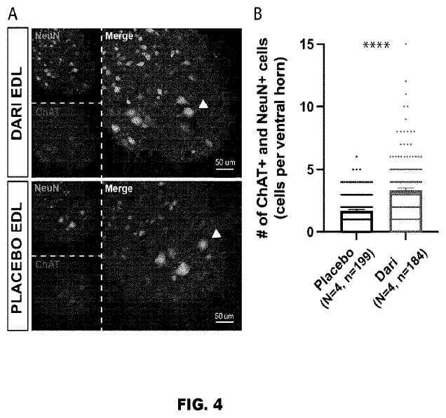

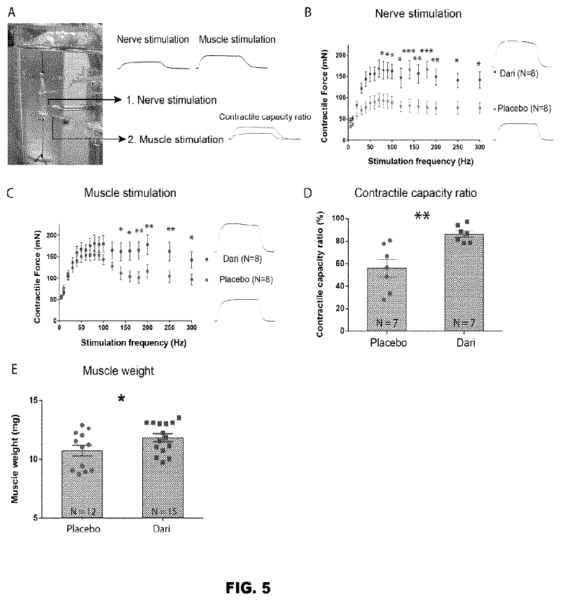

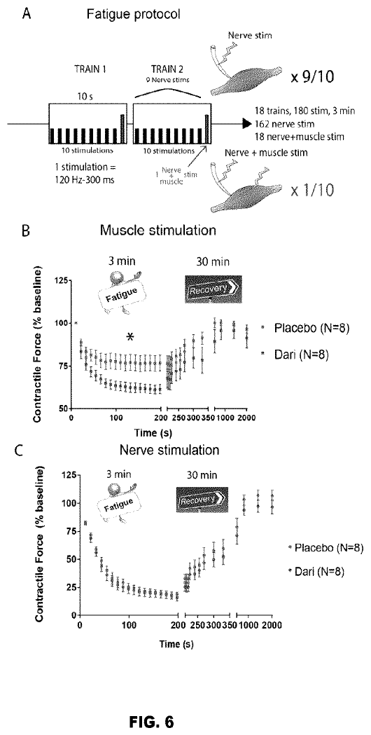

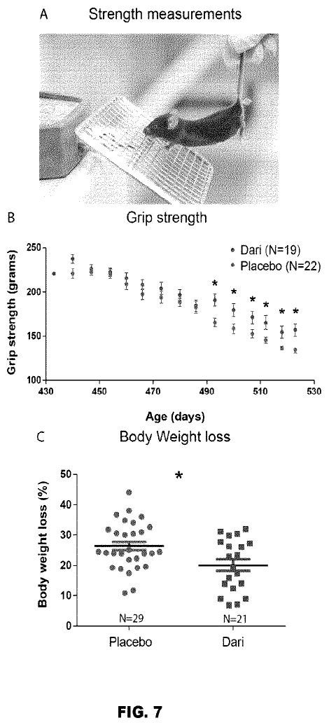

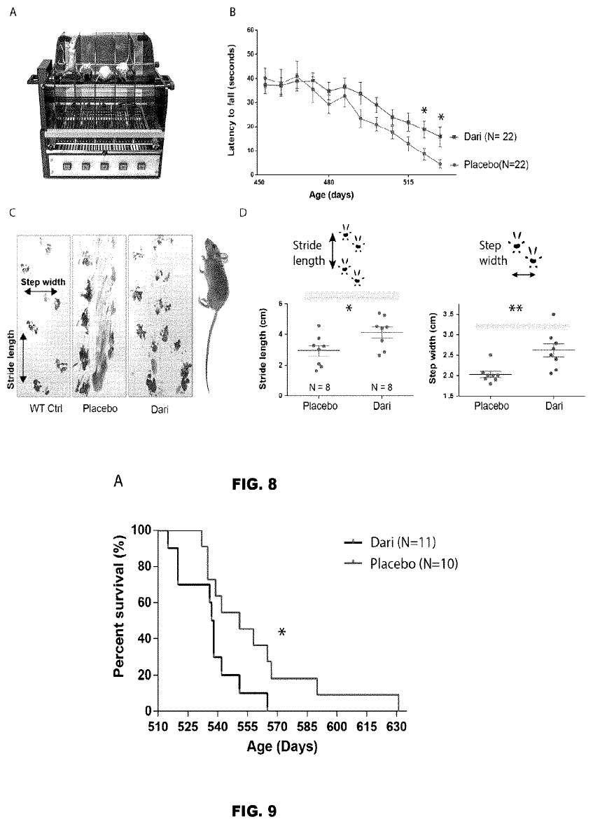

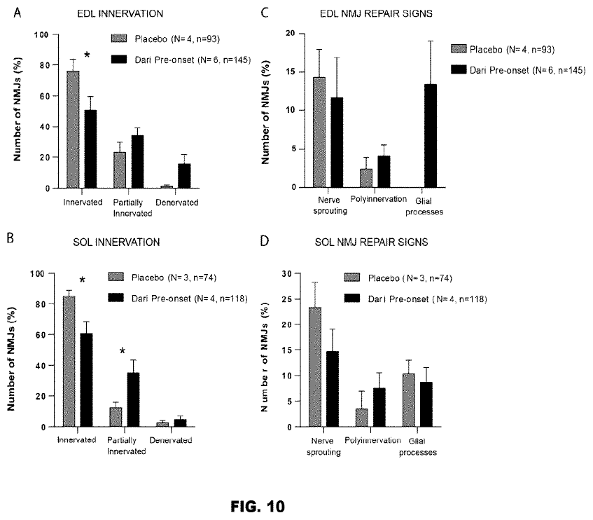

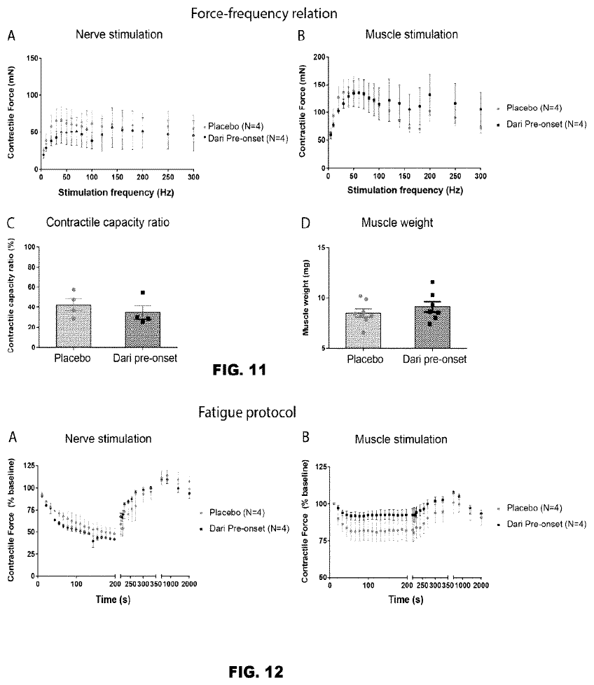

(D) Mice were either treated daily with Darifenacin (10 mg/kg; N=5, n=27) or with placebo (DMSO, N=4, n=19) for 5 days. D) Top: Ca²⁺-responses induced in PSCs by local applications of muscarine (10 μM) on PSCs from placebo (FIG. 1D, a) and from a Darifenacin-treated SOD1^{G37R} mouse (FIG. 1D, b) and from a non-treated WT mouse (FIG. 1D, c). Bottom, dot plot illustrating the mean±S.E.M. of PSCs Ca²⁺ responses (ΔF/F) in both conditions (DMSO: 181.9±20.9; Darifenacin: 106.3±24.5, p=0.0064). The light grey zone represents the mean±S.E.M. of the amplitude of the Ca²⁺-responses induced in PSCs by the agonist in non-treated WT animals. B) Top: Ca²⁺-responses induced in PSCs by local applications of ATP (10 μM) on PSCs from placebo (FIG. 1E, a) and from a Darifenacin-treated SOD1^{G37R} mouse (FIG. 1E, b) and from a non-treated WT mouse (FIG. 1E, c). Bottom, dot plot illustrating the mean±S.E.M. of PSCs Ca²⁺ responses (ΔF/F) in both conditions (DMSO: 478.7±23.3; Darifenacin: 536.5±26.1, p>0.05). The light grey zone represents the mean±S.E.M. of the amplitude of the Ca²⁺-responses induced in PSCs by the agonist in non-treated WT animals.

FIG. 2 Darifenacin treatment at disease onset improves innervation status in the EDL muscle. A) Graphical illustrations of the three types of innervation status included in the morphological analysis: innervated (full coverage of postsynaptic receptors by the nerve terminal), partially innervated (incomplete coverage of the NMJ by the nerve terminal) and denervated (no presynaptic coverage at all). B) Histogram showing the mean±SEM of the percentage of fully innervated, partially innervated and denervated NMJs. C) Confocal images of immunohistochemical labeling of the presynaptic terminal (axon, SV2+NF-M) and postsynaptic nicotinic AChRs (muscle endplate, α-BTX) in the darifenacin-treated EDL (top panel) and the placebo-treated EDL (bottom panel). Arrows indicate denervated NMJs. **, p<0.01, Two-way ANOVA. Scale bar, 10 μm.

FIG. 3 Darifenacin treatment at disease onset increases nerve sprouting, a positive sign of NMJ repair. A) Top panel: Diagram illustrating two signs of NMJ repair: nerve sprouting (large arrowhead) and glial process extension (thin arrowhead). A) Bottom panel and B) Confocal images of immunohistochemical labeling of the NMJ: postsynaptic nicotinic AChRs (muscle endplate, α-BTX) and the merge including in addition the presynaptic terminal (SV2+NF-M) and Schwann cells (S100β) for darifenacin-treated (top panel) and placebo-treated EDL (bottom panel). Note the presence of nerve sprouting (large arrowheads) in the darifenacin-treated animals, and the absence of NMJ repair signs in placebo NMJs, despite ongoing denervation. C) Histogram showing the mean±SEM of the percentage of NMJs presenting nerve sprouting, polyinnervation and/or glial processes in darifenacin-treated vs placebo NMJs. *, p<0.05, Two-way ANOVA. Scale bar, 10 μm.

FIG. 4 Darifenacin treatment at disease onset increases survival of motor neurons in the lumbar spinal cord. A) Confocal images of immunohistochemical labeling of motor neurons in the ventral horn of a lumbar spinal cord section: neuronal-specific nuclear protein (NeuN) and choline acetyltransferase (ChAT) were used to stain spinal cord cross sections of darifenacin-treated (top panel) and placebo-treated mice (bottom panel) at P520. Motor neurons in the ventral horn were counted as α-MN when labeled for both NeuN and ChAT. B) Histogram showing quantification of the number of α-MNs (mean±SEM) in placebo versus darifenacin-treated animals. ****, p<0.0001, Mann-Whitney test. Scale bar, 50 μm.

FIG. 5 Darifenacin treatment at disease onset improves contractile muscle force and contractile capacity ratio. A) Picture of the set up of the muscle force transducer and the nerve and muscle stimulating electrodes used to evoke muscle contractions. Examples of raw data show a muscle contraction elicited by nerve or muscle stimulation, used to calculate the contractile capacity ratio. B-C) Peak twitch force of the EDL generated by nerve stimulations (B) or muscle stimulation at different frequencies (5 Hz-300 Hz) (C) for darifenacin-treated versus placebo-treated animals. D) Histogram showing the mean±SEM of the contractile capacity ratio expressed in percentage, representing the proportion of the peak force generated by nerve stimulation over muscle stimulation (stimulation frequencies between 5 Hz-100 Hz). E) Histogram showing the mean±SEM of EDL muscle weight from placebo-treated and darifenacin-treated mice. *, p<0.05, **, p<0.01, ***, p<0.001, Repeated two-way ANOVA and unpaired t-test.

FIG. 6 Darifenacin treatment at disease onset preserves muscle fatigue properties. A) Diagram illustrating the EDL fatigue protocol, which consists of 18 trains of 10 stimulations elicited at 120 Hz for 300 ms (1 train per second). Nerve stimulations only are used 9 out of 10 bouts and muscle stimulation is superimposed to the nerve stimulation every 10 stimulations. The fatigue protocol is followed by a 30-minute recovery period. B-C) Peak contractile force during the fatigue protocol and the recovery period expressed as the percentage of the initial baseline force generated before the fatigue protocol, for muscle (B) and nerve stimulation (C). Note the higher resistance to fatigue in placebo-treated compared to darifenacin-treated animals. This illustrates a significant alteration of the normal fast-twitch muscle properties that is normally highly fatigable. *, p<0.05, Two-way ANOVA.

FIG. 7 Darifenacin treatment at disease onset slows the deterioration of grip strength and reduces body weight loss. A) Grip strength meter used to monitor the overall strength of mice forelimbs and hindlimbs. B) Evolution of grip strength measurements during the course of the treatment, from P430 to P525 for darifenacin-and placebo-treated animals. C) Percentage of body weight loss (mean±SEM) at P520 for both group of animals. * p<0.05, Unpaired t-test.

FIG. 8 Darifenacin treatment at disease onset ameliorates locomotor function in SOD1 mice. A) Photograph of the Rotarod apparatus used to assess motor function, coordination and balance following an acceleration protocol (4-40 rpm over 300 seconds). B) Latency to fall(s) on the Rotarod of darifenacin-treated versus placebo-treated mice between 450 until 525 days of age. C) Raw data of the gait analysis performed by applying non-toxic ink (forepaws, blue; hind paws, red) on mice paws. Mice walked along a 10 cm-wide corridor and traces were used to calculate the step width (distance between left and right hindlimb traces) and stride length (distance between two subsequent left or right hind-paws). Note the absence of right hind paw traces for placebo-treated animals, indicating onset of hindlimb paralysis, and the relative preservation of the normal WT pattern in darifenacin-treated mice. D) Histograms showing the quantification of stride length and step width for both group of animals (mean±SEM). Grey bar represents the normal WT values for these two parameters. *, p<0.05, **, p<0.01, unpaired t-test.

FIG. 9 Darifenacin treatment at disease onset extends survival. A) Survival curve of darifenacin- and placebo-treated animals. Mice were considered end stage when full hindlimb paralysis and absence of righting reflex within 10 seconds when placed on its left and right side. * p<0.05, Log-rank (Mantel-Cox) test.

FIG. 10 Pre-onset treatment with darifenacin is deleterious to NMJ innervation status in the EDL and SOL muscle at P480. A-B) Histograms showing mean±SEM of the percentage of fully innervated, partially innervated and denervated of placebo-treated (grey) and pre-onset darifenacin-treated mice (black) as determined following immunohistochemistry analysis. The number of fully innervated NMJs is reduced in both muscles for darifenacin-treated animals. C-D) Quantification of EDL and SOL NMJ repair signs expressed as a percentage of the total number of NMJs (mean±SEM): nerve sprouting, polyinnervation and glial process extensions. * p<0.05, Unpaired t-test.

FIG. 11 Pre-onset treatment with darifenacin does not improve contractile muscle force and contractile capacity ratio. A-B) Force-frequency relationship in the EDL muscle following nerve (A) or muscle stimulation (B) at various frequencies (5 Hz-300 Hz) for darifenacin-(black) versus placebo-treated mice (grey). C) Histogram showing the mean±SEM of the contractile capacity ratio expressed in percentage for both group of animals. D) Histogram showing the mean±SEM of EDL muscle weight for both animal groups. No significant differences were observed between the two groups for all parameters.

FIG. 12 Pre-onset darifenacin treatment does not change muscle fatigue properties. Peak contractile force during the EDL fatigue protocol and the 30 minutes recovery period expressed as the percentage of the baseline contractile force generated at the start of the protocol, for nerve stimulation (A) and muscle stimulation (B). Note that both darifenacin-and placebo-treated groups have a high resistance to fatigue for both types of stimulations (WT normal levels would be respectively around 10% and 50% at the end of the fatigue protocol for nerve versus muscle stimulation). This demonstrates a severe loss of normal EDL muscle properties as disease progresses, highlighting the absence of impact of the pre-onset darifenacin treatment.

### DETAILED DESCRIPTION OF THE INVENTION

### Anticholinergic Compound

In a first aspect, the invention provides an anticholinergic compound for use in the treatment of a neuromuscular disorder in a human subject. An anticholinergic compound according to this aspect may be called an anticholinergic compound according to the invention in the context of this application, wherein it is understood that such compound is for use in the treatment of a neuromuscular disorder in a human subject.

An anticholinergic compound is a compound that is able to inhibit the effect of the neurotransmitter acetylcholine at synapses or at neuroeffector junctions such as neuromuscular junctions. Preferably, an anticholinergic compound is a compound that is able to dampen muscarinic acetylcholine receptor activity.

In embodiments, an anticholinergic compound according to the invention is a muscarinic receptor antagonist. A muscarinic receptor, also known as a muscarinic acetylcholine receptor or mAchR, is an acetylcholine receptor that forms a G-protein receptor complex in in the cell membrane of certain neurons and other cells. Muscarinic receptors play several roles in mediating the effect of the neurotransmitter acetylcholine. For example, muscarinic receptor are comprised in pre-synaptic membranes of somatic neurons in neuromuscular juntions, where they are involved in the regulation of acetylcholine release.

Five subtypes of muscarinic receptors, M1-M5, are commonly recognized. This classification originates from their different selectivity towards certain agonists and antagonists. M1, M3 and M5 receptors are coupled with Gq proteins in the cell membrane, while M2 and M4 receptors are coupled with Gi/o proteins in the cell membrane. Without being bound to this theory, genes CHRM1-5 encode for M1-M5 receptors, respectively.

The basal or constitutive activity of a muscarinic receptor is defined as the physical, biological and/or chemical activity of the receptor in the absence of acetylcholine, muscarinic receptor agonists and muscarinic receptor antagonists.

An agonist of a muscarinic receptor, also called a muscarinic receptor agonist, is defined as a compound that increases the physical, biological and/or chemical activity of the receptor when it contacts the receptor. An increased activity means an activity similar to the activity caused by contacting the receptor with acetylcholine.

An antagonist of a muscarinic receptor. also called a muscarinic receptor antagonist, is defined as a muscarinic receptor neutral antagonist or muscarinic receptor negative antagonist.

A muscarinic receptor neutral antagonist is a compound that competes with a muscarinic receptor neutral agonist or with a muscarinic receptor negative antagonist for binding to the receptor, thereby blocking the action of the agonist or the negative antagonist (i.e. increasing or decreasing the activity), while the neutral antagonist does not significantly alter the basal activity of the receptor upon binding alone.

In embodiments, an anticholinergic compound according to the invention is a muscarinic receptor neutral antagonist.

A muscarinic receptor negative antagonist is a compound that decreases the physical, biological and/or chemical activity of the receptor when it contacts the receptor, even in the absence of a muscarinic receptor agonist. A decreased activity means an activity opposite to the activity caused by contacting the receptor with acetylcholine.

In embodiments, an anticholinergic compound according to the invention is a muscarinic receptor negative antagonist.

A muscarinic receptor antagonist is defined as selective for one or more muscarinic receptor subtypes M1, M2, M3, M4 and/or M5 if the effect of the antagonist (blocking an agonist, blocking a negative antagonist or decreasing the activity) is only significant upon contacting a muscarinic receptor of the one or more subtypes, while there is significantly less or no effect upon contacting a muscarinic receptor of another subtype. Hence, is a muscarinic receptor antagonist is said to be selective for muscarinic receptor M3, it is understood that significantly less or no effect is obtained upon contacting the antagonist with a muscarinic receptor of subtype M1, M2, M4 or M5. In this context, significantly less may be at least 2-, 3-, 4-, 5-, 6-, 7-, 8-, 9-, 10-, 20-, 30-, 40-, 50-, 60-, 70-, 80-, 90-, 100-, 200-, 300-, 400-, 500-, 600-, 700-, 800-, 900-, 1000-, 10000-, 100000- or 1000000-fold, or at least 10%, 20%, 30%, 40%, 50%, 60%, 70%, 80%, 90%, or 100% less.

The activity of a muscarinic receptor, preferably of subtype M1, M3 or M5, may be measured using dynamic Ca2+ imaging. These receptors regulate the level of IP3 which then control the release of Ca2+ from internal stores [7].

In embodiments, an anticholinergic compound according to the invention is a muscarinic receptor antagonist which is:
selective for muscarinic receptor M1, or
selective for muscarinic receptor M3, or
selective for muscarinic receptor M5, or
selective for muscarinic receptor M1 and muscarinic receptor M3, or
selective for muscarinic receptor M1 and muscarinic receptor M5, or
selective for muscarinic receptor M3 and muscarinic receptor M5, or
selective for muscarinic receptor M1, muscarinic receptor M3, and muscarinic receptor M5.

In embodiments, an anticholinergic compound according to the invention is a muscarinic receptor antagonist which is:
selective for muscarinic receptor M3, or
selective for muscarinic receptor M1 and muscarinic receptor M3, or
selective for muscarinic receptor M3 and muscarinic receptor M5, or
selective for muscarinic receptor M1, muscarinic receptor M3, and muscarinic receptor M5.

In embodiments, an anticholinergic compound according to the invention is darifenacin, ipratropium bromide, tiotropium bromide, trospium, glycopyrronium, aclidinium, umeclidinium, solifenacin, dicylomine, fesoterodine, flavoxate, glycopyrrolate, propantheline, 1R,2R,4S,5S,7S)-7-[({4-fluoro-2-(thiophen-2-yl)phenyl}carbamoyl)oxy]-9,9-dimethyl-3-oxa-9-azatricyclo[3.3.1.02,4]nonan-9-ium formate (BS46 in [38]), N-(2-[3-([3R]-1-(cyclohexylmethyl)-3-piperidinyl]methylamino)-3-oxopropyl]amino-2-oxoethyl)-3,3,3-triphenyl-propioamide (J-115311 in [39]), 3,3,3-triphenylpropionamide derivatives with one or two amino acid residues between the triphenylpropionic acid moiety and the piperidinylmethylamine moiety ([40]), OrM3 ([41]) or (3R)-3-[[[(3-fluorophenyl)[(3,4,5-trifluorophenyl)methyl]amino]carbonyl]oxy]-1-[2-oxo-2-(2-thienyl)ethyl]-1-azoniabicyclo[2.2.2]octane bromide (CHF 5407 in [41]). Without being bound to this theory, these compounds can be considered muscarinic receptor antagonists.

In embodiments, an anticholinergic compound according to the invention is darifenacin, ipratropium bromide, tiotropium bromide, trospium, glycopyrronium, aclidinium, umeclidinium, 1R,2R,4S,5S,7S)-7-[({4-fluoro-2-(thiophen-2-yl)phenyl}carbamoyl)oxy]-9,9-dimethyl-3-oxa-9-azatricyclo[3.3.1.02,4]nonan-9-ium formate (BS46 in [38]), N-(2-3-([3R]-1-(cyclohexylmethyl)-3-piperidinyl]methylamino)-3-oxopropyl]amino-2-oxoethyl)-3,3,3-triphenyl-propioamide (J-115311 in [39]), 3,3,3-triphenylpropionamide derivatives with one or two amino acid residues between the triphenylpropionic acid moiety and the piperidinylmethylamine moiety ([40]), OrM3 ([41]) or (3R)-3-[[[(3-fluorophenyl)[(3,4,5-trifluorophenyl)methyl]amino]carbonyl]oxy]-1-[2-oxo-2-(2-thienyl)ethyl]-1-azoniabicyclo[2.2.2]octane bromide (CHF 5407 in [41]).

In embodiments, an anticholinergic compound according to the invention is darifenacin, ipratropium bromide, tiotropium bromide, trospium, glycopyrronium, aclidinium, umeclidinium, solifenacin, dicylomine, fesoterodine, flavoxate, glycopyrrolate or propantheline.

In embodiments. an anticholinergic compound according to the invention is darifenacin, ipratropium bromide, trospium, or tiotropium bromide.

In embodiments. an anticholinergic compound according to the invention is darifenacin. Darifenacin may be represented by the following structure:

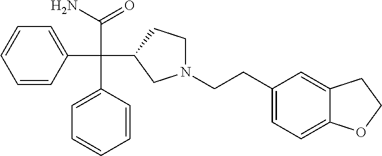 [SMILES: NC(=O)C(c1ccccc1)(c1ccccc1)[C@@H]1CCN(CCc2ccc3c(c2)CCO3)C1]

Preferably, darifenacin is darifenacin hydrobromide. Darifenacin hydrobromide may be represented by the following structure:

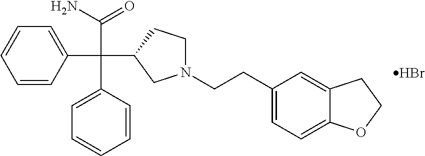 [SMILES: NC(=O)C(c1ccccc1)(c1ccccc1)[C@@H]1CCN(CCc2ccc3c(c2)CCO3)C1 ; Br]

In embodiments, any of the compounds disclosed in the embodiments above may be present as a pharmaceutically acceptable salt therof. In particular, an anticholinergic compound according to the invention is darifenacin or a pharmaceutically acceptable salt thereof.

Examples of pharmaceutically acceptable salts include, without limitation, alkali metal (for example, sodium, potassium or lithium) or alkaline earth metals (for example, calcium) salts; however, any salt that is generally non-toxic and effective when administered to the subject being treated is acceptable. Further salts may include, without limitation: (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, urethane sulfonic acid, ethanesuifonic acid, p-toluenesulfonic acid salicylic acid, tartaric acid citric acid, succinic acid or malonic acid and the like; or (2) salts formed when an acidic proton present in the parent compound either is replaced by a metal ion. e.g., an alkali metal ion, an alkaline earth ion, or an aluminum ion, or coordinates with an organic base such as ethanol amine. diethanolamine, triethanolamine, trimethamine, N-methylglucamine, and the like. Pharmaceutically acceptable salts are well known to those skilled in the art, and any such pharmaceutically acceptable salts may be contemplated in connection with the embodiments described herein.

Acceptable salts may be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, lactate, maiate, maleate, malooate, mesylate, methylsulphate, naphthylate, 2-napsylate, nicotinale, nitrate, orotate, oxalate, palniitate, pamoate, phosphate/hydrogen phosphate/dihydrogen phosphate, saccharate, stearate, succinate, tartrate, tosyiate and trifluoroacetate salts. Suitable base salts of the compounds described herein are formed from bases that form non-toxic salts illustrative, albeit nonlimiting, examples include the arginine. benzathine, calcium, choline, diethylamine, diolamine, glycine. lysine, magnesium, meglumine, olamine, potassium, sodium, tromethamine and zinc salts. Hemisalts of acids and bases may also be formed, for example, hemisulphate and hemicalcium salts.

### Composition

An anticholinergic compound according to the invention may be advantageously administered as a composition.

Therefore in a further aspect, there is provided a composition for use in the treatment of a neuromuscular disorder in a human subject, said composition comprising an anticholinergic compound according to the invention. A composition according to this aspect may be called a composition according to the invention in the context of this application, wherein it is understood that such composition is for use in the treatment of a neuromuscular disorder in a human subject.

In an embodiment, a composition according to the invention is a pharmaceutical composition. In an embodiment, said pharmaceutical compositon comprises at least one pharmaceutically acceptable carrier or excipient.

In an embodiment, compositions according to the invention are pharmaceutical compositions comprising an active therapeutic agent (i.e., an anticholinergic compound according to the invention) and one or more of a variety of other pharmaceutically acceptable components. See REMINGTON: THE SCIENCE AND PRACTICE OF PHARMACY (21st Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline. Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, com oil, peanut oil. cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the anticholinergic compound according to the invention. Compositions according to the invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol. and the like; and (3) metal chelating agents. such as citric acid, ethylenediamine tetraacetic acid (EDTA), sorbitol, tartaric acid, phosphoric acid, and the like.

Compositions according to the invention may also comprise isotonicity agents, such as sugars, polyalcohols, such as mannitol, sorbitol, glycerol or sodium chloride in the compositions.

Compositions according to the invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The anticholinergic compounds according to the invention may be prepared with carriers that will protect the compounds against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the anticholinergic compound according to the invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required partide size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, anticholinergic compounds according to the invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient. An exemplary composition comprises an scFv at about 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

The anticholinergic compounds or compositions according to the invention of the present invention can be administered by parenteral, topical. oral or intranasal means for therapeutic treatment. Intramuscular injection (for example, into the arm or leg muscles) and intravenous infusion are preferred methods of administration of the molecules of the present invention. In some methods, such molecules are administered as a sustained release composition or device, such as a Medipad™ device (Elan Pharm. Technologies, Dublin, Ireland). In some methods, the anticholinergic compounds or compositions according to the invention are injected directly into a particular tissue, for example intracranial injection. In one embodiment, a pharmaceutical composition according to the invention is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

In embodiments, a composition according to the invention is formulated as a once-a-day extended release tablet for oral use comprising darifenacin, preferably as darifenacin hydrobromide. Preferably, the compositions comprise one or more of the following excipients: dibasic calcium phosphate anhydrous, hypromellose, magnesium stearate, titanium dioxide, iron oxide yellow, iron oxide red, PEG 400 and/or talc. In an embodiment, the composition according to the invention is known as ENABLEX™. ENABLEX™ is formulated as a 7.5 mg or 15 mg darifenacin (as darifenacin hydrobromide).

### Therapeutic Use

The therapeutic use of a compound according to the invention induces a mechanism that promotes NMJ (neuromuscular junction) stability and/or repair, which is attractive for the treatment of any neuromuscular disease, especially wherein such NMJ is affected, such as ALS or a disease that mimics ALS (‘ALS-like disease’).

In embodiments, a anticholinergic compound according to the invention is for use in the treatment of a neuromuscular disorder in a human subject, wherein the neuromuscular disorder is characterized by an impaired neuromuscular transmission and/or an NMJ denervation.

In embodiments, an impaired neuromuscular transmission may be characterized by at least one of:
a. muscarinic overexcitability,
b. motor neuron death,
c. impaired synaptic transmission and
d. NMJ denervation.

In an embodiment, an impaired for neuromuscular transmission may be characterized by a poor motor performance, a decreased grip strength, the poor contractile properties of a muscle at the NMJ, the poor resistance to fatigue of the muscle, a decreased muscle weight.

In an embodiment, a neuromuscular disorder is analyzed or assessed or diagnosed via electrophysiological assessment; pharmacodynamic assessment: the level of neurofilaments (e.g. neurofilament light chain (NFL)) in blood serum, plasma and/or cerebrospinal fluid (CSF); or NMJ biopsies.

In an embodiment, the treatment of the neuromuscular disorder results in an improvement, relative to a human subject not being treated with an anticholinergic compound or a composition comprising an anticholinergic compound according to the invention, in an electrophysiological assessment; in a pharmacodynamic assessment; in neurofilaments (e.g. neurofilament light chain (NFL)) in blood serum. plasma and/or cerebrospinal fluid (CSF); or in NMJ biopsies of the treated human subject.

The therapeutic effects of the an anticholinergic compound or composition according to the invention, as described elsewhere herein may be assessed by the same methods.

A neuromuscular disorder may be selected from the group consisting of: amyotrophic lateral sclerosis (ALS), spinal muscular atrophy (SMA), myasthenia gravis (MG), congenital myasthenia, Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. A preferred neuromuscular disorder is ALS or a disease that mimics ALS (‘ALS-like disease’). Examples of diseases that mimic ALS include: spinal muscular atrophy (SMA), myasthenia gravis (MG), congenital myasthenia, Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. A more preferred neuromuscular disorder is ALS.

In an embodiment, an anticholinergic compound according to the invention is used for the treatment of a neuromuscular disorder and/or a neurodegenerative disorder, e.g. ALS.

In an embodiment, an anticholinergic compound according to the invention is administered at disease onset or within 1, 2, 3, 4, 5, 6, 7, 8, 9, 10, 11, 12, 13, 14, 15, 16, 17, 18, 19, 20, or 21 days; within 1, 2, 3, 4, 5, 6, or 7 weeks; or within 1, 2, 3, 4, 5, 6, 7, 8, 9, 10, 11, or 12 months following disease onset. In an embodiment, the anticholinergic compound is administered at disease onset or within one week after disease onset. Surprisingly, atractive results were obtained when the anticholinergic compound was administered at disease onset or as soon as possible after disease onset. It is to be understood by the skilled person that the anticholinergic compound is preferably not merely used to reduce or diminish a symptom associated with the neuromuscular disorder (such as ALS). In other words, the treatment with the anticholinergic compound is preferably not merely a symptomatic treatment. In an embodiment, the anticholinergic compound is not used to reduce or diminish urinary urgency. In an embodiment, the anticholinergic compound is not used to reduce or diminish urinary urgency in a neuromuscular disease such as ALS.

In a preferred embodiment, disease onset includes at least one of the symptoms selected from the group consisting of: muscle twitches, muscle cramps, spasticity, muscle weakness, slurred and/or nasal speech, difficulty chewing or swallowing, dysphagia, dysarthria and dyspnea. In a more preferred embodiment, the disease is ALS and disease onset includes at least one of the symptoms selected from the group consisting of: muscle twitches, muscle cramps, spasticity, muscle weakness, slurred and/or nasal speech, difficulty chewing or swallowing, dysphagia, dysarthria and dyspnea.

Disease onset may be assessed by a physician or veterinarian. In an embodiment, beginning of weight loss is considered as disease onset.

In an embodiment, an anticholinergic compound according to the invention is administered at disease onset, or within 1, 2, 3, 4, 5, 6, 7, 8, 9, 10, 11, 12, 13, 14, 15, 16, 17, 18, 19, 20, or 21 days; within 1, 2, 3, 4, 5, 6, or 7 weeks; or within 1, 2, 3, 4, 5, 6, 7, 8, 9, 10, 11, or 12 months following disease onset, but prior to a diagnosis of the disease. In preferred embodiments, the administration is between diagnosis and 1, 2, 3, 4, 5, 6, 7, 8, 9, 10, 11, 12, 13, 14, 15, 16, 17, 18, 19, 20, or 21 days; within 1, 2, 3, 4, 5, 6, or 7 weeks; or within 1, 2, 3, 4, 5, 6, 7, 8, 9, 10, 11, or 12 months prior to diagnosis.

In an embodiment a human subject or a patient who has been diagnosed with a neuromuscular disorder such as one of those disclosed above, the anticholinergic compound according to the invention is administered to such patient in an amount sufficient to cure, treat, or at least partially arrest the symptoms of the disease (as adduced by biochemical, histologic and/or behavioral assessment), including its complications and intermediate pathological phenotypes in development of the disease. In some embodiments, the administration of the therapeutic molecules of the present invention reduces or eliminates the neuromuscular disorder.

An effective dose or amount of an anticholinergic compound according to the invention or a composition according to the invention refers to an amount sufficient, at dosages and for periods of time necessary, to achieve an intended biological effect or a desired therapeutic result including, without limitation, clinical results. The phrase “therapeutically effective amount” when applied to an anticholinergic compound according to the invention or a composition according to the invention is intended to denote an amount of the compound or composition that is sufficient to ameliorate, palliate, stabilize, reverse, slow or delay the progression of a disorder or disease state, or of a symptom of the disorder or disease. In an embodiment, the method of the present invention provides for administration of the anticholinergic compound according to the invention in combinations with other compounds. In such instances, the effective dose is the amount of the combination sufficient to cause the intended biological effect.

Effective doses of the anticholinergic compound according to the invention, for the treatment of the above-described conditions may vary depending upon many different factors, including means of administration, target site, physiological state of the patient, other medications administered. Treatment dosages are typically titrated to optimize their safety and efficacy. On any given day that a dosage is given, the dosage of the anticholinergic compound according to the invention may range from about 0.0001 to about 100 mg/kg, and more usually from about 0.01 to about 20 mg/kg, of the patient's body weight. For example, dosages can be 1 mg/kg body weight or 10 mg/kg body weight or within the range of 1-10 mg/kg body weight. Exemplary dosages thus include: from about 0.1 to about 10 mg/kg body weight, from about 0.1 to about 5 mg/kg body weight, from about 0.1 to about 2 mg/kg body weight, from about 0.1 to about 1 mg/kg body weight, for instance about 0.15 mg/kg body weight, about 0.2 mg/kg body weight, about 0.5 mg/kg body weight, about 1 mg/kg body weight, about 1.5 mg/kg body weight, about 2 mg/kg body weight, about 5 mg/kg body weight, or about 10 mg/kg body weight.

A physician or veterinarian having ordinary skill in the art may readily determine and prescribe the effective amount of a anticholinergic compound according to the invention or composition according to the invention required. For example, the physician or veterinarian could start doses of the compound or composition at levels lower than that required in order to achieve the desired therapeutic effect and gradually increase the dosage until the desired effect is achieved. In general, a suitable daily dose of a composition of the present invention will be that amount of the compound which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the anticholinergic compound according to the invention to be administered alone, it is preferable to administer the anticholinergic compound according to the invention as a pharmaceutical composition as described above.

For therapeutic purposes, the anticholinergic compound according to the invention and the composition according to the invention are usually administered on multiple occasions. Intervals between single dosages (e.g., a bolus or infusion) can be weekly, monthly, or yearly. In some methods, dosage is adjusted to achieve a plasma concentration of 1 ng/ml up to 1000 μg/ml, preferably 1-1000 μg/mL, more preferably 25-300 μg/mL. Alternatively, the therapeutic molecules of the present invention can be administered as a sustained release formulation, in which case less frequent administration is required. Dosage and frequency vary depending on the half-life of the compound in the patient.

The term “treatment” or “treating” as used herein means ameliorating, slowing or reversing the progress or severity of a disease or disorder, or ameliorating, slowing or reversing one or more symptoms or side effects of such disease or disorder. For purposes of this invention, “treatment” or “treating” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state. delay or slowing of the progression a disease or disorder state, amelioration or palliation of a disease or disorder state, and remission of a disease or disorder, whether partial or total, detectable or undetectable.

Accordingly, in an embodiment, an anticholinergic compound according to the invention or a composition according to the invention is for use in the treatment of a neuromuscular disorder in a human subject, wherein said treatment results in a stabilization of said disorder. The stabilization may be for at least 1, 2, 3, 4, 5, 6, 7, 8, 9, 10, 11 or 12 months or at least 1, 2 or 3 years. Each of the therapeutic effects further characterized herein could be seen as a stabilization of the disorder.

In an embodiment, the use of an anticholinergic compound according to the invention or a composition according to the invention exhibits a therapeutic effect on the treated human subject defined herein. Such a therapeutic effect may be at least one of the effects disclosed below.

A therapeutic effect of an anticholinergic compound according to the invention or a composition according to the invention may be the increase of the number or percentage of fully innervated NMJ, maintenance of the number or percentage of fully innervated NMJ (disease stabilization/disease progression stabilization), the decrease of the number or percentage of fully denervated NMJ, an improvement of the reliability of synaptic transmission, a prevention/stabilization or even a reduction/decrease of motor neuron death. Each of these features could be assessed using techniques known to the skilled person such as staining of AChR using an a-bungarotoxin antibody, presynaptic labelling and quantifying innervation by fluorescent confocal microscopy, EMG single fibre EMG, electrophysiology of single synapses, staining of motor neuron cell bodies in bone marrow specific regions. All these assays have been described in Cantor S et al 2018 (Elife, 2018;7: e34375).

A therapeutic effect of an anticholinergic compound according to the invention or a composition according to the invention may be the improvement of the motor performance and/or grip strength of the treated subject. The motor performance and grip strenght of a treated subject may have been considered to have been improved when such motor perfromance or grip strength may have been increased by at least 10%, 20%, 30%, 40%, 50%, 60%, 70%, 80%, 90%, or 100% in an experiment using the anticholinergic compound according to the invention by comparison with the same experimental setting without the compound. The motor performance (or grip strenght) of a treated subject may be assessed using assays known to the skilled person. The experimental part discloses some exemplary methods. In this context, the subject may be an animal.

A therapeutic effect of an anticholinergic compound according to the invention or a composition according to the invention may be the improvement of the contractile properties of a muscle at the NMJ of the treated subject. The contractile properties of a muscle of a treated subject may have been considered to have been improved when such contractile properties may have been increased of at least 10%, 20%, 30%, 40%, 50%, 60%, 70%, 80%, 90%, or 100% in an experiment using the anticholinergic compound according to the invention by comparison with the same experimental setting without the compound. The contractile properties of the muscle of a treated subject (at the NMJ) may be assessed using assays known to the skilled person. The experimental part discloses some exemplary methods. In this context, the subject may be an animal.

A therapeutic effect of an anticholinergic compound according to the invention or a composition according to the invention may be the improvement of the resistance to fatigue of a muscle at the NMJ of the treated subject. The resistance to fatigure of a muscle of a treated subject may have been considered to have been improved when such fatigue properties may have been improved of at least 10%, 20%, 30%, 40%, 50%, 60%, 70%, 80%, 90%, or 100% in an experiment using the anticholinergic compound according to the invention by comparison with the same experimental setting without the compound. The fatigure properties of the muscle of a treated subject (at the NMJ) may be assessed using assays known to the skilled person. The experimental part discloses some exemplary methods. In this context, the subject may be an animal.

A therapeutic effect of an anticholinergic compound according to the invention or a composition according to the invention may be the induction of an increase of the muscle weight at the NMJ of the treated subject. The muscle weight of a treated subject may have been considered to have been improved when such weight may have been increased of at least 10%, 20%, 30%, 40%, 50%, 60%, 70%, 80%, 90%, 100% in an experiment using the anticholinergic compound according to the invention by comparison with the same experimental setting without the compound. The experimental part discloses some exemplary methods. In this context, the subject may be an animal.

A therapeutic effect of an anticholinergic compound according to the invention or a composition according to the invention may be characterized by the improvement of the quality of life or the delay in the apparition of the deterioration of the quality of life of a treated subject. The quality of life may be quantified by the weight of the subject. The improvement of the quality oflife or the delay in the apparition of the deterioration of the quality of life may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected quality of life (or the expected apparition of the deterioration of the quality of life) of a subject suffering from the same condition and having not been treated with an anticholinergic compound according to the invention In this context, the subject may be an animal.

A therapeutic effect of an anticholinergic compound according to the invention or a composition according to the invention may be characterized by the lifespan of a treated subject. The extension may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected lifespan of a subject suffering from the same condition and having not been treated with an anticholinergic compound according to the invention. In this context. the subject may be an animal.

A therapeutic effect of an anticholinergic compound according to the invention or a composition according to the invention may be the reduction (‘dampening’) of the muscarinic activity of perisynaptic Schwann cells (PSC), the NMJ repair. It is demonstrated in the experimental part that an anticholinergic compound of the invention such as darifenacin specifically reduces (“dampens”) the muscarinic activity of PSC. The compound reduces (dampens) the hyperexcitability of PSC in the context of a neuromuscular discorder. This compound specifically acts on the muscarinic receptor. This compound does not seem to have any effect on the purigenic receptor expressed on PSC.

NMJ repair may be the induction or increase of nerve sprouting and/or the increase of the innervation status of the NMJ. Each of these effects may be assessed using techniques known to the skilled person.

In the context of the invention, an induction or increase of nerve sprouting (or of the innervation status of the NMJ) may have been assessed when the induction of nerve sprouting at the NMJ (or of the innervation status of the NMJ) is increased of at least 10%, 20%, 30%, 40%, 50%, 60%, 70%, 80%, 90%, 100% in an experiment using the anticholinergic compound according to the invention by comparison with the same experimental setting without said compound. Nerve sprouting or innervation status may be assessed using immunohistochemistry on nerve-muscle preparations. The experimental part discloses how to obtain such nerve-muscle preparations.

In the context of the invention, the reduction of the muscarinic activity of PSC (or the reduction of the muscarinic hyperexcitability or overexcitability) may have been assessed when such activity (or such hyperexcitability or overexcitability) has been reduced of at least 10%, 20%, 30%, 40%, 50%, 60%, 70%, 80%, 90%, 100% in an experiment using the anticholinergic compound according to the invention by comparison with the same experimental setting without said compound.

Accordingly, in an embodiment, an anticholinergic compound, or a composition is for use according to the invention, wherein the administration of said anticholinergic compound or composition to said human subject results in one or more of the following therapeutic effects:
an increase of the number or percentage of fully innervated NMJ in the subject, maintenance of the number or percentage of fully innervated NMJ in the subject, the decrease of the number or percentage of fully denervated NMJ in the subject, an improvement of the reliability of synaptic transmission, a prevention, stabilization or reduction of motor neuron death in the subject; and/or
an improvement of the motor performance and/or grip strength of the subject; and/or
an improvement of the contractile properties of a muscle at the NMJ of the subject; and/or
an improvement of the resistance to fatigue of a muscle at the NMJ of the subject; and/or
an induction of an increase of the muscle weight at the NMJ of the subject; and/or
an improvement of the quality of life of the delay in the apparition of the deterioration of the quality of life of the subject; and/or
a reduction of the muscarinic activity (or the reduction of the muscarinic hyperexcitability or overexcitability) of perisynaptic Schwann cells (PSC) in the subject, or of the NMJ repair in the subject.

In a further aspect, there is provided a method for the prevention and/or treatment of a neuromuscular disease and/or disorder and/or condition comprising administering to a subject in need thereof a compound according to the invention or a composition according to the invention. All features of this method have been defined earlier herein.

In a further aspect, there is provided the use of a compound according to the invention or a composition according to the invention in the prevention and/or treatment of a neuromuscular disease and/or disorder and/or condition in a subject in need thereof. All features of this method have been defined earlier herein. In a further aspect, there is provided a use of a compound according to the invention or a composition according to the invention in the manufacture of a medicament for the prevention and/or treatment of a neuromuscular disease and/or disorder and/or condition in a subject in need thereof. All features of this use have been defined earlier herein.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of a VHH according to the invention if the composition as such would exhibit similar characteristic/activity/functionality as said VHH.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The terms ‘disorder’ and ‘disease’ are used herein interchangeably.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### EXAMPLES

### Example 1: Target Engagement: Darifenacin Successfully Reduces PSC Muscarinic Hyperexcitability

### Example 1.1: Target Engagement: Darifenacin Successfully Reduces PSC Muscarinic Hyperexcitability

First, we determined the target engagement of darifenacin and determined the best dosage compatible with a moderate level of receptors blockade. To this end, we verified if the darifenacin treatment successfully dampened mAChRs Ca²⁺ responses in SOD1 mice. A group of mice received doses of either the placebo, darifenacin at 10 mg/kg or 15 mg/kg as described in the methods for 30 days. This dosage regime was chosen based on the pre-clinical data available for the use of darifenacin in the over-reactive bladder treatment.

Mice were then sacrificed, and muscles dissected with their innervation and placed in a physiological recording chamber. We performed Ca²⁺ imaging on the SOL muscle to evaluate PSCs synaptic detection of neurotransmission elicited by motor nerve stimulation (50 Hz-5 sec) or local applications of the mAChRs agonist muscarine for the treated and the placebo groups. We observed a dose-dependent diminution of Ca²⁺ response elicited by local muscarine applications, with most responses being abolished at the highest dosage (FIG. 1A-B). PSCs muscarinic activation was greatly abolished at a dosage of 15 mg/kg as shown by an important reduction in the percentage of Ca²⁺ responses and in the amplitude of the Ca²⁺ responses that was significantly reduced (FIG. 1A). At the dosage of 10 mg/kg, the percentage of responding cells was about 50% of placebo and while the average amplitude of Ca²⁺ responses was also 50% of the placebo amplitude. Importantly, darifenacin had no effect on the purinergic signaling of PSCs as indicated by the unaltered Ca²⁺ responses elicited by the local application of ATP (FIG. 1A, C). This shows that darifenacin did not alter the other main receptor system responsible for PSCs activity.

We opted for the dosage at 10 mg/kg since the primary goal is to restore PSCs activity by dampening their muscarinic activity close to WT levels. This reduced PSC muscarinic excitability demonstrates target engagement of our treatment. In addition, darifenacin was detected in the blood with levels consistent with these pre-clinical data (data not shown). These were both essential steps to pursue the study.

### Example 1.2: Darifenacin Target Engagement: Darifenacin Treatment Dampens Muscarinic Hyperexcitability in SOD1 Mice

Darifenacin (10 mg/kg diluted in DMSO) or DMSO was given orally for 5 days in SOD1^{G37R} mice (˜P400). Preparations of SOL muscles and their innervation were dissected and pinned in a Sylgard-coated experimental chamber filled with an oxygenated Ree's solution (in mM, all Sigma), as follows: 110 NaCl, 5 KCl, 1 MgCl₂, 25 NaHCO₃, 2 CaCl₂, 11 glucose, 0.3 glutamic acid, 0.4 glutamine, 5 BES (N,N-Bis(2-hydroxyethyl)-2-aminoethanesulfonic acid sodium salt), 0.036 choline chloride, and 4.34×10⁻⁷ cocarboxylase. Nerve-muscle preparations were incubated for 90 min (2×45 min) in a preoxygenated saline solution containing 10 μM fluo-4AM (Invitrogen) and 0.02% pluronic acid (Invitrogen). After the loading, nerve muscle preparations were constantly perfused with a warm (26-28° C.) oxygenated Ree's solution (95% O₂, 5% CO₂). Basel level of fluorescence was always set within the same range of arbitrary units, using the same software and hardware settings. Changes in fluorescence were measured over PSC soma and expressed as follows: % ΔF/F=(F-Fᵣₑₛₜ)/Fᵣₑₛₜ×100. Experiments were discarded when focus drift occurred. To prevent muscle contractions, postsynaptic cholinergic receptors were blocked with D-tubocurarine chloride (2.5 μg/ml, Sigma). PSC Ca²⁺ responses were elicited by local application of agonists using a brief, small pulse of positive pressure (20 PSI, 150 ms) generated by a Picospritzer II (Parker Instruments) applied on a glass pipette (5 MΩ, ˜2.5 μm-tip diameter) positioned in proximity of the cells. Adenosine 5-triphosphate (ATP, 10 mM) or muscarine (10 μM, Sigma), known to respectively activate PSC purinergic and muscarinic receptors, was dissolved in the same Ree's solution used for the experiment. PSCs at several NMJs were imaged per muscle and a recovery of at least 20 min was allowed between each application when several applications were performed on the same cells.

These data suggest that Darifenacin specifically acts on muscarinic receptors at PSC and dampens muscarinic hyperexcitability observed in SOD1^{G37R} mice. Indeed, Darifenacin treatment in SOD1^{G37R} mice reduced the PSC Ca²⁺ response induced by local applications of muscarine to similar levels as non-treated wild type mice (FIG. 1D). Whereas, no difference in the PSC Ca²⁺ response induced by local applications of ATP (activating PSC purinergic receptors) was observed, when comparing SOD1^{G37R} mice treated with Darifenacin or DMSO and non-treated wild type mice (FIG. 1E).

### Example 2: Beneficial Effects of Darifenacin Treatment at Disease Onset

### Example 2.1: Darifenacin Preserves NMJ Innervation Status

According to our hypothesis, dampening PSCs excitability with darifenacin should facilitate NMJ functions. To test this possibility, we first investigated NMJ structure and integrity by performing immunohistochemistry to monitor the impact of the darifenacin treatment on NMJ innervation. Darifenacin treatment was initiated at disease onset as described in the method section.

We characterized EDL innervation status, a vulnerable fast-twitch muscle in ALS [3, 25-27], by classifying NMJs into fully innervated, partially innervated and denervated NMJs (FIG. 2A). NMJs from placebo-treated animals presented a significant loss of presynaptic coverage and a higher percentage of denervated NMJs than darifenacin-treated mice (FIG. 2B; Dari 5.6±1.8%, N=7, n=293; Placebo 50.9±13.7%, N=7, n=307; p<0.001, Bonferroni post-hoc test). In addition, NMJs from darifenacin treated mice were significantly more fully innervated than placebo-treated mice, with 70.1±4.2% vs 22.0±8.9% innervated NMJs in the placebo group (FIG. 2B; Dari, N=7, n=293; Placebo, N=7, n=307; p<0.01, Bonferroni post-hoc test). Most NMJs presented a complete presynaptic coverage and a preserved NMJ normal structure as opposed to a higher presence of denervated NMJ in placebo-treated SOD1 mice (see example FIG. 2C). There was no change in the number of partially innervated NMJs (FIG. 2B; Dari 24.2±4.0%, N=7, n=293; Placebo 27.1±6.4%, N=7, n=307; p>0.05, Bonferroni post-hoc test).

### Example 2.2: Darifenacin Increases Nerve Sprouting

In normal conditions, nerve injury or neurotransmitters release interruption results in a reduced mAChRs signaling at denervated NMJs that leads to PSCs extension of glial processes [14, 15, 37]. These processes can subsequently support and guide nerve terminal sprouting from innervated NMJs toward denervated ones [16, 17, 28]. Nerve terminal cannot extend sprouting without a glial support. Hence, dampening PSCs muscarinic activation with darifenacin should also restore their ability to engage in NMJ repair as shown by the presence of glial processes that guide and support nerve terminal sprouting [4, 7] (FIG. 3A).

Indeed, in EDL muscles from darifenacin-treated mice, there was a significant increase in NMJs presenting nerve sprouting profiles, which is indicative of synapse remodeling in the EDL (FIG. 3B-C; Dari 21.5±4.3%, N=7, n=293; Placebo 7.6±3.7%, N=7, n=307; p<0.01, Bonferroni post-hoc test). These changes are particularly important considering that this fast-twitch muscle is composed mainly of fast fatigable motor units that show a weak competence in nerve sprouting following injury or ALS [2, 16, 18]. Interestingly, there was no significant difference in the number of glial processes alone in the darifenacin-treated in comparison to placebo, indicative that most processes were occupied by nerve terminal sprouts and consistent with effective glial guidance mechanisms (FIG. 3C; Dari 2.2±1.8%, N=7, n=293; Placebo 2.2±1.7%, N=7, n=307; p>0.05, Bonferroni post-hoc test).

Lastly, we examined for the presence of polyinnervation as another sign of NMJ repair. However, no differences were observed between the darifenacin-treated animals and the ones that received the placebo (FIG. 3C; Dari 6.4±2.8, N=7, n=293; Placebo 3.9±2.4%,, N=7, n=307; p>0.05, Bonferroni post-hoc test).

### Example 2.3: Darifenacin Reduces Motor Neuron Death

We assessed next if the chronic treatment with darifenacin would be beneficial to motor neuron survival. Indeed, although darifenacin poorly crosses the blood-brain-barrier, we posit that stabilizing and preserving NMJ innervation status would reduce an important stress factor on motor neurons[6], thus enhancing their ability to manage other stress factors occurring in the spinal cord. We prepared lumbar spinal cords sections as previously described[6] and labeled MNs with the cholinergic neuronal marker ChAT and the general neuronal nuclei marker NeuN to identify α-MNs. Neurons positive for both markers located in the ventral horn were counted as α-MNs. As shown in FIG. 4 (A-B), darifenacin-treated animals presented a significantly higher MN count per ventral horn than the placebo group (Dari N=4, n=184; Placebo N=4, n=199; p<0.0001, Mann-Withney test). Hence, stabilizing NMJs indirectly enhanced motor neuron survival.

### Example 2.4: Darifenacin Improves Neuromuscular Contractile Muscle Force and NMJ Efficacy

Improved NMJ innervation and motor neuron survival are strong indicators that the muscle functions should also be improved by the darifenacin treatment. To investigate this, we used a musde force transducer to measure the force generated by EDL muscles elicited by stimulation of the motor nerve and/or by the direct muscle stimulation (FIG. 5A). With this system, stimulation of the motor nerve at various frequencies elicits muscle contraction through NMJ efficacy, reflecting the strength of contractile fibers associated with innervated NMJs only. Muscle stimulation, on the contrary, depolarizes all muscle fibers and reflect maximal twitch force of all the muscle, independent of the innervation status.

This method is especially useful to characterise diseases like ALS presenting NMJ and muscular deficits [29, 30].

We first performed a standard protocol of stimulation to generate a force frequency curve (5 Hz-300 Hz) to characterize NMJ efficacy and muscle force following the darifenacin chronic treatment. Muscle force generated by the contractions elicited the stimulation of the motor nerve and NMJ activation, EDL from darifenacin-treated group demonstrated significantly higher twitch forces (138.0±9.1 mN) than EDL from the placebo group (75.9±4.1 mN) during the protocol. These values represent respectively 55% and 30% of the WT mean twitch force value (FIG. 5B; Dari N=8, Placebo N=8; p<0.01, Repeated Two-way ANOVA). Contractile forces generated differed between the two groups from 70 Hz to 300 Hz (FIG. 5B; Dari N=8, Placebo N=8; p<0.05, Two-way ANOVA, Bonferroni post hoc test). In the case of the direct muscle stimulation, significant differences between the darifenacin-treated mice and the placebo group were observed at higher frequencies (between 140 Hz and 300 Hz). This is indicative of the preservation of the fast-twitch properties of the EDL (FIG. 5C; Dari N=8, Placebo N=8; p<0.05 et p<0.01, unpaired t-tests at each frequency).

We next determined the proportion of the muscle capacity that is used by the neuromuscular system upon nerve stimulation. This was expressed as the contractile capacity ratio calculated as follow:

$$\frac{{Force}_{Nerve}}{{Force}_{Muscle}} \times 100$$

This ratio is at 100% in WT mice, indicating that the neuronal control of the muscle recruits 100% of its contractile capacity. Hence, by improving NMJ innervation, resulting in an increase in force generated, we posit that this ratio should be higher in EDL muscles from darifenacin-treated animals compared to placebo. As shown in FIG. 5D, the ratio was significantly higher in treated mice, with 86.1±2.8% compared to 55.7±7.8% for control mice (Dari N=7, Placebo N=7; p<0.01, unpaired t-test). Interestingly, this increase in contractile force and contractile capacity ratio was observed with a better preservation of the EDL muscle weight in treated animals (FIG. 5E; Dari 11.79±0.34 mg, N=8, n=16; Placebo 10.7±0.46 mg, N=8, n=15; p<0.05, Wilcoxon matched-pair signed rank tests).

### Example 2.5: Danfenacin Preserves Muscle Fatigue Properties

In addition to the force generated, a muscle is also characterised by its resistance to fatigue. For instance, fast-twitch muscles composed of a majority of fast fatigable motor units like the EDL, is their great fatigability[31]. In ALS, alteration at the type of innervation (from fast to slow twitch) and in the properties of muscles themselves alter the fatigue properties, rendering them more resistant. Since the treatment by darifenacin significantly preserved muscle strength, we next investigated the resistance to fatigue of the EDL. We used a fatigue stimulation protocol, followed by a 30 min recovery period (FIG. 6A). While EDL muscles from the placebo-treated group showed an atypical resistance to fatigue when directly stimulated, EDL muscles from darifenacin-treated mice showed a level of fatigue that is more typical for this type of fast twitch muscle (FIG. 6B: Dari N=8, Placebo N=8; p<0.01, Repeated two-way ANOVA). However, we found no difference in the rate of fatigue of muscle contractions generated by motor nerve stimulation (FIG. 6C; Dari N=8, Placebo N=8; p>0.05, Repeated two-way ANOVA). This is likely an indication of a pathological higher resistance to fatigue acquired during disease progression [29, 30, 32], but also potentially reflects the higher rate of denervation observed in the placebo-treated mice, as denervated fibers are only solicited during muscle stimulation and do not fatigue during nerve stimulations. There was no difference in the rate of recovery following the fatigue protocol between the darifenacin-treated mice and the placebo-treated ones (FIG. 6B-C; Dari N=8, Placebo N=8; p>0.05, Repeated two-way ANOVA).

### Example 2.6: Danfenacin Improves Locomotor Function and Gait

Next, we wanted to investigate if this increase in NMJ efficacy and muscle function by the darifenacin treatment resulted in an amelioration in locomotor function and general strength of the animal. Mice treated with darifenacin were still walking around the cage, exploring their environment while the placebo-treated mice showed a severe hind limb paralysis. A number of tests were performed to quantify the improvement of the motor behaviour by the darifenacin treatment.

First, we monitored the grip strength to assess if the darifenacin treatment ameliorated the general strength of the animals (FIG. 7A). Mice of the darifenacin and placebo groups began the trial with a similar grip strength force. However, darifenacin-treated mice performed better than the ones in the placebo group as shown by the larger grip strength they generated from P490 until the end of the preclinical trial (FIG. 7B; Dari N=19; Placebo N=22, Two-way ANOVA, Bonferroni post-hoc test, p<0.05 and p<0.01).

Second, since the darifenacin treatment had a major improvement on the NMJ morphological integrity, motor neurons survival, muscle properties and general motor functions, we next tested whether this had an impact on the general condition of the mice. To this end, we monitored the changes in the body weight of the animals, a metric that is directly related to the progression of the disease and survival whereby animals present an important gradual body weight loss after symptoms onset. As shown in FIG. 7C, body weight loss was reduced in the darifenacin-treated group compared to the placebo-treated mice (Dari 20.0±1.9% N=21; Placebo 26.4±1.5% N=29; p<0.05, Two-way ANOVA, Bonferroni post-hoc test).

Third, the motor performance, balance and coordination, were measured using a standard acceleration protocol on the Rotarod (FIG. 8A), which is known to reveal ALS motor deficits as disease progresses [33]. FIG. 8B shows that the performance of darifenacin-treated and placebo-treated mice progressively declined as revealed by a shorter latency to fall of the Rotarod, showing the expected progression of ALS motor phenotype. However, the motor performance of darifenacin-treated mice was increased compared to placebo-treated mice during the progression of the disease, particularly when approaching the end stage, with a significantly higher score obtained at age P520 and P525 (FIG. 8A-B; Dari N=22; Placebo N=22; p<0.05, Unpaired t-test). Indeed, at this late symptomatic stage, most of the placebo mice were no longer able to perform the test while more than half of the darifenacin-treated group were still able to stand for several seconds on the rotating rod (data not shown).

Finally, we aimed to better characterize the walking pattern of the mice following treatment. We measured the gait by performing a footprint analysis using inked paws. We quantified stride length and step width, two key parameters that are gradually reduced during disease progression as gait becomes impaired, as can be seen in the examples in FIG. 8C. Stride length represents the distance between two consecutive steps performed by the same hindlimb and was increased at P520 for our mice treated with darifenacin (FIG. 8D; Dari 4.13±0.36 cm, N=8; Placebo 2.94±0.35 cm, N=8; p<0.05, Unpaired t-test). Step width. which represent the distance between left and right hind-paws, was also reduced in the placebo group as compared to the darifenacin-treated group at P520 (FIG. 8D; Dari 2.62±0.16 cm, N=8; Placebo 2.02±0.07 cm, N=8: Unpaired t-test, p<0.01).

### Example 2.7: Darifenacin Improves the General Condition and Extends Mice Survival

Finally, to investigate if treatment with darifenacin increased survival, we initiated a survival cohort of mice starting at disease onset (P450) that received the same regime of dosage except that mice received continuous treatment until reaching the end stage, corresponding at the level 5 of the disease (see appendix 1). Treatment with darifenacin successfully extended the survival of mice with a median lifespan of 551 days in comparison to a median survival of 537 days for placebo treated mice (FIG. 9A; Dari N=10; Placebo N=11, p<0.05, Mantel-Cox test).

### Example 3: Inefficient or Deleterious Effects of Darifenacin Treatment Before Disease Onset (Pre-Onset)

The initial elaboration of the muscarinic hypothesis [7, 12] in ALS by our team implied that the dampening of mAChRs should help PSCs to perform NMJ repair and contribute to a healthy innervation status, without any consideration for the disease stage. Our initial observation by Arbour et al. (2015) revealed the hyper-muscarinic activation of PSCs was already present early in the disease process (P120 and P380). Hence, our first attempt was to start the darifenacin treatment at an age before disease onset (P400) that corresponded to the one reported in our initial publication. However, we did not anticipate that the time during disease progression for administration of the treatment was in fact a key factor modulating its efficacy.

### Example 3.1: Pre-Onset Chronic Treatment with Darifenacin is Deleterious to NMJ Innervation

To investigate the impact of the darifenacin pre-onset treatment on NMJ morphology, we first performed immunohistochemistry to characterize NMJ innervation status and NMJ repair signs on the EDL and the SOL muscles of treated mice from P400 until P480 and their SOD1 controls.

In the vulnerable fast-twitch EDL muscle, surprisingly, complete innervation was significantly reduced in darifenacin-treated compared to placebo-treated mice (FIG. 10A: Dari 50.4±9.2%, N=6, n=145; Placebo 75.9±7.7%, N=4, n=93; p<0.05, Two-way ANOVA, Bonferroni post-hoc test). This implies that significantly more NMJs were either partially or completely denervated in darifenacin-treated animals, although the trend was not significant when considered separately (FIG. 10A; Dari 33.9±5.1% and 15.7±5.9%, N=6, n=145; Placebo 23.06.8% and 1.1±1.1%, N=4, n=93; p>0.05, Two-way ANOVA, Bonferroni post-hoc test). The same tendency was observed in the more resistant slow-twitch SOL muscle: darifenacin-treated NMJs were significantly less fully innervated than control mice (FIG. 10B; Dari 60.5±8.2%, N=4, n=118; Placebo 85.0±3.8%, N=3, n=74; p<0.05, Bonferroni post-hoc test). In parallel, an increase in partial innervation in the SOL was detected in darifenacin-treated animals (FIG. 10B; Dari 34.9±8.6%, N=4, n=118; Placebo 12.47±3.5%, N=3, n=74; p<0.05, Bonferroni post-hoc test). This suggests that our treatment, unlike what was suggested in our initial work (Arbour et al., 2015), when started before symptoms onset, not only did not improve NMJ innervation status but seems to exacerbate the disease process by accelerating the progress of NMJ denervation, completely refuting our original idea.

Next, we investigated the presence of signs of NMJ repair to evaluate if the pre-onset treatment impacts the glial mechanisms that are regulated by their muscarinic activation, thus central to our muscarinic hypothesis. To this end, we analysed three signs of NMJ repair, namely nerve sprouting, polyinnervation and glial processes, as described previously (FIG. 2A). We found no evidence of significant difference in the darifenacin-treated group and the placebo-treated group for any of the parameters, neither in the SOL or the EDL (FIG. 10C-D: Dari N=4, n=118; Placebo N=3, n=74; p<0.05, Two-way ANOVA, Bonferroni post-hoc test). However, in the EDL muscle, which normally shows low competence for NMJ plasticity [2, 4], glial process extensions were seen in 5 out of 6 darifenacin-treated animals but were totally absent in the placebo-treated mice. However, this was not associated with any other sign of positive impact on reinnervation of the treatment, including glial process extensions. This lack of glial process extension is important as it implies that the nerve terminal guidance that they normally exert to facilitate reinnervation and functional remodeling of the synapse did not take place.

### Example 3.2: Pre-Onset Treatment with Darifenacin Do not Improve Neuromuscular Function

Even though the pre-onset treatment with darifenacin was altering negatively the NMJ innervation, we nonetheless evaluated neuromuscular function in these cohorts of animals. Indeed, there was a possibility that the remaining NMJs compensated for the innervation with high efficacy, thus resulting in some improvements in muscle functions. Hence, to fully appreciate pre-onset therapy impact, we started the darifenacin treatment prior to symptoms appearance (P400) until the late symptomatic stage (P520). Using the muscle force transducer, we measured the peak EDL contractile force generated following nerve or muscle direct stimulation following a standard stimulation protocol, to evaluate the force-frequency relationship across a wide range of frequencies (5 Hz-300 Hz). When stimulating the nerve or the muscle, no difference was observed in the peak muscle force generated at any frequencies between the two groups of animals (FIG. 11A-B; Dari N=4; Placebo, N=4; p>0.05, unpaired t-tests). These measurements allowed us to calculate the contractile capacity ratio, as described earlier, to compare the twitch force generated by neuronal control of muscular contraction to the direct muscle maximal twitch force. Ratios were similar for darifenacin-treated mice as compared to placebo-treated mice, with respectively 34.64±6.74% and 42.45±6.36% (FIG. 11C; Dari N=4; Placebo N=4; p>0.05, unpaired t-test). The fact that contractile capacity ratios were strongly reduced in both groups furthermore demonstrated that darifenacin treatment did not improve NMJ efficacy when started at the pre-symptomatic stage. Finally, EDL muscle weight did not differ between darifenacin-treated mice and placebo-treated mice (FIG. 11D; Dari 9.1±0.55 mg, N=4, n=7; Placebo 8.46±0.41 mg, N=4, n=8; p>0.05, unpaired t-test), suggesting again the absence of benefits of the pre-onset treatment. Of note, two darifenacin-treated animals were discarded of the study because they developed an early onset of forelimb paralysis and reached the final stage at an early age (P490).

Finally, we investigated the fatigue properties of the EDL muscle following darifenacin pre-onset treatment. As mentioned earlier, fast-twitch muscles like the EDL are very strong but are normally highly fatigable [31]. However, in ALS, a gradual resistance to fatigue of fast-twitch muscles have been unraveled, following progression of disease pathogenesis [29, 32]. We used a fatigue stimulation protocol (180 trains at 120 Hz-300 ms), followed by a 30 min recovery period (see FIG. 6A) to investigate treatment impact on EDL fatigue properties. EDL muscles from darifenacin-treated mice and placebo-treated group showed a similar atypical resistance to fatigue following nerve stimulation (FIG. 12A; Dari N=4, Placebo N=4; p>0.05, Repeated two-way ANOVA). As shown in FIG. 12, no difference was observed between the two groups in the rate of fatigue generated by muscle stimulation (FIG. 12B; Dari N=4, Placebo N=4; p>0.05, Repeated two-way ANOVA). The strong resistance of the muscle to fatigue probably reflected the high rate of denervation of the EDL muscle at the symptomatic stage in both darifenacin-and placebo-treated mice. The rate of recovery following the fatigue protocol was also comparable between the darifenacin-treated mice and the placebo-treated ones (FIG. 12A-B; Dari N=4, Placebo N=4; p>0.05, Repeated two-way ANOVA).

As a whole, an early pre-onset treatment with darifenacin failed to improve NMJs innervation and any muscle functions. This strongly highlights the fact that the timing of administration of the darifenacin therapy is of crucial importance to exert beneficial effects on ALS disease progression. Unlike what could have been expected from our initial work, darifenacin treatment to dampen mAChRs in ALS to rescue NMJ innervation, muscle functions and locomotion must be performed during a critical time window starting at disease onset to a timepoint after symptom onset.

### Example 4: Conclusion

The data presented herein show that darifenacin has great potential for the treatment of ALS. Our extensive work was based on the novel idea that PSCs need to present a muscarinic excitability that is adapted to the specific state of the NMJ to efficiently perform their crucial role in NMJ repair and reinnervation [7,12]. Hence, we postulated that the hyper-muscarinic excitability observed in ALS could prevent PSCs from efficiently performing their normal functions and limit NMJ repair. This was the basis of our original muscarinic hypothesis, in which we suggested that reducing PSC muscarinic activity will have a positive impact on NMJ innervation status and motor functions in ALS, representing an imaginative and original therapeutic strategy targeting PSCs to improve quality of life of ALS patients. Our preclinical trial allowed us to confirm this hypothesis but only when the selective M3 AChRs antagonist darifenacin treatment is started at disease onset. Hence, the treatment is started at a clinically relevant time, making this approach a valuable avenue for the treatment of ALS. Interestingly, darifenacin has few and mild side effects in part since it does not cross the blood brain barrier [19-24]. Hence, the therapeutic potential of darifenacin treatment is significant while posing minimal risk of side effects.

### Example 5: Methods

### Animals

Mice overexpressing the human mutated SOD1^{G37R} transgene, line 29, were obtained from The Jackson Laboratory and bred at our animal facilities on a C57BL/6 background. This mouse model is a late onset, slowly progressing model of ALS that recapitulates the human phenotype of the disease. Characterization of this strain phenotype has been previously published in several ALS studies [4, 7, 18, 34]. All experiments were performed in accordance with the guidelines of the Canadian Council of Animal Care and the Comité de déontologie animale of Université de Montréal.

### Preclinical Trial Design

The pre-clinial trial design was made according to guidelines for preclinical animal research in ALS/MND [35]. Male mice from the SOD1^{G37R} background and their WT littermates were included in the preclinical study. Mice were randomly assigned to the experimental or the placebo group. Treatment, behavior monitoring, experiments and results analysis were done blindly. Standard ALS behavioral measurements were performed biweekly to measure disease progression in the various study groups. This includes rotarod test. grip strength measurements, weight measurements and tail suspension test to assess hindlimb extension reflex.

A set of neurological scores (level 1 to 5, Appendix 1) was used to determine the onset of symptoms and the progression and severity of symptoms during disease progression. Treatment was started at disease onset and continued for different time periods, depending on the goal of the experiments. The timing of treatment at disease onset aligns with our ultimate goal to achieve clinical impact since the lack of predictive biomarkers means that an ALS diagnosis in patients is still only achieved after clinical symptom presentation.

Onset of disease was assessed by the beginning of weight loss[36] and appearance of tremor, representing a neurological score of 1. Darifenacin or Placebo administration was started at P450 (P440-460). which is the mean age of onset in the SOD1^{G37R} [4]. For the experimental group, darifenacin was given orally at a dose of 10 mg/kg, 5 days a week, diluted in DMSO. Mice receiving the placebo received the same oral dose of DMSO only, without darifenacin. Two primary endpoints were considered in the study. For the regular cohort, endpoint was at the late symptomatic stage, at the age of P520 (P515-535). For the survival cohort, endpoint was end stage of the disease. corresponding in an absence of righting reflex within 10-15 seconds when placed on its left and right side and full hindlimb paralysis (neurological score, level 5).

### Rotarod Acceleration Protocol

Motor coordination, strength and balance were assessed using a rotarod (TSE Rotarod, TSE Systems Gmbh, Germany). Animals were placed onto a rotating wheel at a starting speed of 4 rpm, increasing to 40 rpm in 300 seconds. Mice had two attempts per session to remain on the rotarod during the acceleration protocol, and the longest latency to fall was counted.

### Gait Analysis

Gait was assessed using the footprint test. Paws were inked with a nontoxic paint (fore-paws in blue and hind-paws in red), mice were placed on a restricted pathway on a white paper sheet (30 cm long, 6 cm wide) and free to walk along the walkway. Five footsteps from each run were measured for the following parameters (cm): (1) stride length (distance between front and hind limbs) and (2) Step width (distance between left and right paws). The mean value of each set of outcomes was calculated for statistical analysis.

### Nerve-Muscle Preparations

Preparations of Extensor Digitorum Longus (EDL) and Soleus (SOL) muscles and their innervating nerve were dissected in oxygenated Ree's solution (in mM), as follows: 110 NaCl, 5 KCl, 1 MgCl₂, 25 NaHCO₃, 2 CaCl₂, 11 glucose, 0.3 glutamic acid, 0.4 glutamine, 5 BES (N,N-Bis(2-hydroxyethyl)-2-aminoethanesulfonic acid sodium salt), 0.036 choline chloride, and 4.34×10⁻⁷ cocarboxylase. After dissection, nerve muscle preparations were constantly perfused with oxygenated Ree's solution (95% O₂, 5% CO₂).

### Calcium Imaging of PSCs to Determine Target Engagement

Muscles and their innervation were dissected and pined in a physiological recording chamber. Imaging of PSCs intracellular Ca²⁺ was performed as described previously [7, 18]. Nerve-muscle preparations were incubated for 90 min (2×45 min) in a preoxygenated physiological saline solution containing 10 μM fluo-4AM. Ca²⁺ elevations in PSCs where via two stimulating approaches. First, direct application of agonists muscarine and ATP (10 μM in pipette) from a micropipette positioned at proximity of the cells. The percentage of responding cells and the amplitude of the relative changes in Ca²⁺ (% ΔF/F) were determined following darifenacin chronic treatment.

### Measurements of Neuromuscular Properties

The EDL and SOL nerve-muscle preparations were attached vertically to a fixed force transducer (model 402 A-500 mN, Aurora Scientific Inc.) using surgical threads. The preparations were attached at the tendons level to the transducer at one extremity and to an adaptable hook at the opposite extremity. A platinum reference electrode was then juxtaposed ot the muscle, positioned near an extremity of the muscle, close to a tendon. To stimulate the muscle, a second platinum electrode was juxtaposed at the other extremity of the muscle. To elicit muscle contractions from motor nerve and neuromuscular activity, the tibial nerve (SOL) or the deep peroneal nerve (EDL) motor nerve was suctioned into an electrode made of PE tubing and filled with Ringer's solution. Hence, this system was designed to elicit muscle contractions from both muscle and/or nerve stimulations. Neuromuscular contractile basal force responses were elicited by a single supra-maximal square-wave of 500 mV, 0.1 ms pulse imposed on the motor nerve. Muscle contractile basal force responses were elicited by square pulse stimulation of 15 V, 1 ms. Optimal muscle length was determined by gradually stretching the muscle until maximal contractile force output was attained.

Force-frequency curve: Nerve and muscle stimulations were performed to generate a standard force-frequency curve. Alternate nerve and muscle stimulations were performed at various frequencies (5 Hz, 10 Hz, 20 Hz, 30 Hz, 40 Hz, 50 Hz, 60 Hz, 70 Hz, 80 Hz, 90 Hz, 100 Hz, 120 Hz, 140 Hz, 160 Hz, 180 Hz, 200 Hz, 250 Hz and 300 Hz) and the force generated monitored. There was a 2-minute rest period between each stimulation.

Muscle fatigue: The fatigue protocol is illustrated in FIG. 6A. The fatigue protocol was adapted to each muscle owing to the differences in their intrinsic properties. For the EDL, fatigue was tested using a bout of 180 nerve stimulations for a duration of 300 ms, elicited at a frequency of 120 Hz. The rest period between each stimulation was 700 ms, for a total protocol duration of 3 min. Muscular stimulations were super-imposed to nerve stimulations every 10 stimulations (18 simultaneous nerve-muscle stimulations), to evaluate muscular reserve. The fatigue protocol for the SOL consisted of a bout of 300 nerve stimulations for 500 ms at 50 Hz, with a rest period of 600 ms between stimulations, for a total duration of 5 min 30. Muscular stimulations were super-imposed to nerve stimulations every 10 stimulations (30 simultaneous nerve-muscle stimulations).

Each fatigue protocol was followed by a 30 min recovery period during which muscular and neuromuscular contractile force were measured at the following latencies (after the end of the fatigue protocol): 5 s, 10 s, 15 s, 30 s, 45 sec, 1 min, 1.5 min, 2 min, 2.5 min, 5 min, 10 min, 20 min and 30 min.

### Spinal Cord Cross-Sections and Muscle Immunohistochemistry

Motor neuron counts: Mouse were perfused transcardially and lumbar spinal cord were subsequently dissected, cryoprotected in a 30% sucrose solution and frozen using dry ice [6]. Floating lumbar spinal cord cryosections were processed as previously described [4, 6]. Spinal cords cryosections were double stained against Choline Acetyl-Transferase (ChAT: 1:100; Goat; Millipore, Canada; AB144P) and the neuronal marker Neuronal Nuclei (NeuN; 1:300; Mouse IgG1; Millipore; MAB377). ChAT and NeuN positive cells were identified as α-MNs[4]. A minimin of twenty lumbar spinal cord sections per animal were analysed and double-positive cells from each ventral horns were quantified.

Neuromuscular innervation: Immunostaining was performed on EDL and SOL muscle previously described [6]. Briefly, NMJs were labeled to visualise the axon terminal, Schwann cells and the post-synaptic receptors. The presynaptic element was labeled using an anti-synaptic vesicular protein 2 (SV2, 1:2000, Developmental Studies Hybridoma Bank) and an anti-neurofilament M, (NF-M, 1:1000, Rockland Immunochemicals Inc). PSCs and Schwann cells were labeled with anti-S100β antibody (1:4, Z0311, Dako) and post-synaptic receptors with Alexa594-conjugated-α-BTX (1.33 μg/ml, Molecular Probes, Fischer Scientific, Canada).

Observations and image acquisition were obtained using a Zeiss LSM 880 confocal microscope, with a 63× oil immersion objective (N.A. 1.4) or an Olympus FV 1000, with either a 63× oil immersion objective or a 20× water immersion objective (respectively N.A. 0.95 and 0.90). No image manipulations were performed after acquisition, except for brightness and contrast adjustments for figure presentation.

### Statistics

Results are represented as the mean±SEM where the number of animals is identified as N (number of replicates) and the number of NMJs or number of cells for calcium imaging experiments is represented by n (number of observations). Unpaired t tests were used in most cases where two different groups were compared. In addition, two-way ANOVA with post hoc Bonferroni multiple comparison test was used to compare the values obtained for NMJ innervation status and NMJ repair in the Dari vs placebo group. Repeated one-way ANOVA with post hoc Bonferroni multiple comparison test was used to compare the values obtained at various frequencies or over time from the same animals in the treatment vs the placebo group. Survival curves were compared using the log-rank (Mantel-Cox) test. The confidence level used in the study was 95% (α=0.05). All analyses were made with GraphPad 8 software (Prism).

### Example 6: Phase II Trial with Darifenacin in ALS Patients

We will perform a randomized, double-blind, placebo-controlled, single centre, trial to test the safety, tolerability, pharmacokinetics and pharmacodynamics of darifenacin in adults with ALS. In addition, the study will provide preliminary data on the effect of darifenacin on function, and on target engagement with respect to muscle strength/integrity, to inform subsequent trial designs. Potential biomarkers will also be investigated.

### Specific Aims

Primary outcomes: While darifenacin is already approved for the treatment of overactive bladder, its safety and tolerability in patients with ALS has not been tested. Hence, the primary outcome of the study is the safety and tolerability of darifenacin in patients with ALS. Tolerability is defined as reaching the target dose and remaining on the study drug until the planned discontinuation at the final 10th visit. We will also perform a pharmacokinetic analysis of darifenacin during the course of the treatment.

Secondary outcomes: Four secondary outcomes will be studied.
1. Physical function: will be assessed using the Revised Amyotrophic Lateral Sclerosis Functional Rating Scale (ALSFRS-R).
2. Measurements of muscle function: Our pre-clinical trial data on mouse models indicated that our treatment maintained neuromuscular innervation, leading to better muscle and motor functions. Hence, as part of the secondary outcomes, we will use a series of direct reporters of muscle properties and function. We will explore muscle strength variation from baseline, using hand-held dynamometry (HHD). We will also examine muscle properties using electrical impedance myography (EIM).
3. Biometrics: We will monitor the body weight of patients to calculate their body mass index (BMI) since a higher BMI is a strong predictor of disease progression and survival.
4. Respiratory function measures: Forced Vital Capacity (FVC) will be measured by a trained respiratory therapist and will be used to assess the respiratory capacity of the study population at baseline and at the end of the treatment period.

Exploratory outcomes: The novelty of our approach resides in targeting NMJs in ALS. Hence, we propose to include exploratory outcomes based on neuromuscular-related measures that are validated for other diseases but not commonly used to diagnose and characterize ALS. The approaches described below will allow us to directly assess the quality and status of neuromuscular junction innervation, and thus target engagement by darifenacin.
1. Analysis of muscle and NMJ properties using electrophysiological markers: we will exploit electrophysiological assessments that are normally used in other neuromuscular conditions. This includes threshold tracking nerve conduction studies (TTNCS), compound muscle action potentials (CMAP) and single fiber electromyography (SFEMG).
2. Pharmacodynamic assessments and biomarkers: We will monitor the plasma levels of neurofilaments and neuromuscular proteins, particularly extracellular matrix proteins.
3. NMJ biopsies: Unlike treatments that target CNS elements that are physically inaccessible, our approach targets neuromuscular junctions, a structure that is readily accessible through standard medical procedures. This direct access will allow us to monitor the efficacy of the treatment, providing a direct readout of target engagement.

### REFERENCES

1. Fischer, L. R., et al., Amyotrophic lateral sclerosis is a distal axonopathy: evidence in mice and man. Exp Neurol, 2004. 185(2): p. 232-40.
2. Frey, D., et al., Early and selective loss of neuromuscular synapse subtypes with low sprouting competence in motoneuron diseases. J Neurosci, 2000. 20(7): p. 2534-42.
3. Pun, S., et al., Selective vulnerability and pruning of phasic motoneuron axons in motoneuron disease alleviated by CNTF. Nat Neurosci, 2006. 9(3): p. 408-19.
4. Tremblay. E., E. Martineau, and R. Robitaille, Opposite Synaptic Alterations at the Neuromuscular Junction in an ALS Mouse Model: When Motor Units Matter. J Neurosci, 2017. 37(37): p. 8901-8918.
5. Yang, C., et al., Mutant PFN1 causes ALS phenotypes and progressive motor neuron degeneration in mice by a gain of toxicity. Proc Natl Acad Sci U S A. 2016. 113(41): p. E6209-E6218.
6. Martineau, E., et al., Dynamic neuromuscular remodeling precedes motor-unit loss in a mouse model of ALS. Elife, 2018. 7.
7. Arbour, D., et al., Early and persistent abnormal decoding by glial cells at the neuromuscular junction in an ALS model. J Neurosci, 2015. 35(2): p. 688-706.
8. Robitaille. R., B. S. Jahromi, and M. P. Charlton, Muscarinic Ca2+ responses resistant to muscarinic antagonists at perisynaptic Schwann cells of the frog neuromuscular junction. J Physiol, 1997. 504 (Pt 2): p. 337-47.
9. Rochon, D., I. Rousse, and R. Robitaille, Synapse-glia interactions at the mammalian neuromuscular junction. J Neurosci, 2001. 21(11): p. 3819-29.
10. Ko, C. P. and R. Robitaille, Perisynaptic Schwann Cells at the Neuromuscular Synapse: Adaptable, Multitasking Glial Cells. Cold Spring Harb Perspect Biol, 2015. 7(10): p. a020503.
11. Wright, M. C., et al., Distinct muscarinic acetylcholine receptor subtypes contribute to stability and growth, but not compensatory plasticity, of neuromuscular synapses. J Neurosci, 2009. 29(47): p. 14942-55.
12. Arbour, D., C. Vande Velde, and R. Robitaille, New perspectives on amyotrophic lateral sclerosis: the role of glial cells at the neuromuscular junction. J Physiol, 2017. 595(3): p. 647-661.
13. Son, Y. J. and W. J. Thompson, Nerve sprouting in muscle is induced and guided by processes extended by Schwann cells. Neuron, 1995. 14(1): p. 133-41.
14. Georgiou, J., et al., Synaptic regulation of glial protein expression in vivo. Neuron, 1994. 12(2): p. 443-55.
15. Georgiou, J., R. Robitaille, and M. P. Charlton, Muscarinic control of cytoskeleton in perisynaptic glia. J Neurosci, 1999. 19(10): p. 3836-46.
16. O'Malley, J. P., M. T. Waran, and R. J. Balice-Gordon, In vivo observations of terminal Schwann cells at normal. denervated, and reinnervated mouse neuromuscular junctions. J Neurobiol, 1999. 38(2): p. 270-86.
17. Reynolds, M. L. and C. J. Woolf. Terminal Schwann cells elaborate extensive processes following denervation of the motor endplate. J Neurocytol, 1992. 21(1): p. 50-66.
18. Martineau. E., et al., Properties of Glial Cell at the Neuromuscular Junction Are Incompatible with Synaptic Repair in the SOD1(G37R) ALS Mouse Model. J Neurosci, 2020. 40(40): p. 7759-7777.
19. Haab, F., Darifenacin in the treatment of overactive bladder. Drugs Today (Barc), 2005. 41(7): p. 441-52.
20. Chapple, C. R., Darifenacin: a novel M3 muscarinic selective receptor antagonist for the treatment of overactive bladder. Expert Opin Investig Drugs, 2004. 13(11): p. 1493-500.
21. Chapple, C., et al., A pooled analysis of three phase III studies to investigate the efficacy, tolerability and safety of darifenacin, a muscarinic M3 selective receptor antagonist, in the treatment of overactive bladder. BJU Int, 2005. 95(7): p. 993-1001.
22. Haab, F., L. Stewart, and P. Dwyer, Darifenacin, an M3 selective receptor antagonist, is an effective and well-tolerated once-daily treatment for overactive bladder. Eur Urol, 2004. 45(4): p. 420-9; discussion 429.
23. Callegari, E., et al., A comprehensive non-clinical evaluation of the CNS penetration potential of antimuscarinic agents for the treatment of overactive bladder. Br J Clin Pharmacol, 2011. 72(2): p. 235-46.
24. Kay, G. G. and U. Ebinger. Preserving cognitive function for patients with overactive bladder: evidence for a differential effect with darifenacin. Int J Clin Pract, 2008. 62(11): p. 1792-800.
25. Valdez, G., et al., Shared resistance to aging and ALS in neuromuscular junctions of specific muscles. PLoS One, 2012. 7(4): p. e34640.
26. Hegedus, J., C. T. Putman, and T. Gordon. Time course of preferential motor unit loss in the SOD1 G93A mouse model of amyotrophic lateral sclerosis. Neurobiol Dis, 2007. 28(2): p. 154-64.
27. Hegedus, J., et al., Preferential motor unit loss in the SOD1 G93A transgenic mouse model of amyotrophic lateral sclerosis. J Physiol, 2008. 586(14): p. 3337-51.
28. Son, Y. J. and W. J. Thompson, Schwann cell processes guide regeneration of peripheral axons. Neuron, 1995. 14(1): p. 125-32.
29. Rizzuto, E., et al., Measuring Neuromuscular Junction Functionality in the SOD1(G93A) Animal Model of Amyotrophic Lateral Sclerosis. Ann Biomed Eng, 2015. 43(9): p. 2196-206.
30. Dibaj, P., E. D. Schomburg, and H. Steffens, Contractile characteristics of gastrocnemius-soleus muscle in the SOD1G93A ALS mouse model. Neurol Res, 2015. 37(8): p. 693-702.
31. Kanning, K. C., A. Kaplan, and C. E. Henderson, Motor neuron diversity in development and disease. Annu Rev Neurosci, 2010. 33: p. 409-40.
32. Atkin, J. D., et al., Properties of slow-and fast-twitch muscle fibres in a mouse model of amyotrophic lateral sclerosis. Neuromuscul Disord, 2005. 15(5): p. 377-88.
33. Knippenberg. S., et al., Significance of behavioural tests in a transgenic mouse model of amyotrophic lateral sclerosis (ALS). Behav Brain Res, 2010. 213(1): p. 82-7.
34. Wong. P. C. and D. R. Borchelt, Motor neuron disease caused by mutations in superoxide dismutase 1. Curr Opin Neurol, 1995. 8(4): p. 294-301.
35. Ludolph, A. C., et al., Guidelines for preclinical animal research in ALS/MND: A consensus meeting. Amyotroph Lateral Scler, 2010. 11(1-2): p. 38-45.
36. Boillee, S., et al., Onset and progression in inherited ALS determined by motor neurons and microglia. Science, 2006. 312(5778): p. 1389-92.
37. Perez-Gonzalez, A. P. et al., Functional adaptation of glial cells at neuromuscular junctions in response to injury. In revision.
38. Liu, Hongtao, et al. “Structure-guided development of selective M3 muscarinic acetylcholine receptor antagonists.” Proceedings of the National Academy of Sciences 115.47(2018): 12046-12050.
39. Ami, Nozomi, et al. “Selective M3 muscarinic receptor antagonist inhibits small-cell lung carcinoma growth in a mouse orthotopic xenograft model.” Journal of pharmacological sciences 116.1 (2011): 81-88.
40. Sagara Y, Sagara T, Mase T, Kimura T, Numazawa T, Fujikawa T, et al. Cyclohexylmethylpiperidinyttriphenylpropioamide: a selective muscarinic M 3 antagonist discriminating against the other receptor subtypes. J Med Chem. 2002;45:984-987.
41. Moulton, Bart C., and Allison D. Fryer. “Muscarinic receptor antagonists, from folklore to pharmacology; finding drugs that actually work in asthma and COPD.” British journal of pharmacology 163.1 (2011): 44-52.

1. An anticholinergic compound for use in the treatment of a neuromuscular disorder in a human subject.

2. An anticholinergic compound for use according to claim 1, wherein the anticholinergic compound is a muscarinic receptor antagonist.

3. An anticholinergic compound for use according to claim 2, wherein the muscarinic receptor antagonist is selective for muscarinic receptor M1 and/or muscarinic receptor M3 and/or muscarinic receptor M5.

4. An anticholinergic compound for use according to claim 3, wherein the muscarinic receptor antagonist is selective for muscarinic receptor M3.

5. An anticholinergic compound for use according to claim 4, wherein the muscarinic receptor antagonist is darifenacin, ipratropium bromide, trospium or tiotropium bromide, preferably wherein the muscarinic receptor antagonist is darifenacin.

6. A composition comprising an anticholinergic compound for use according to any one of claims 1 to 5 for use in the treatment of a neuromuscular disorder in a human subject.

7. A composition for use according to claim 6, wherein the composition is a pharmaceutical composition comprising at least one pharmaceutically acceptable carrier or excipient.

8. An anticholinergic compound for use according to any one of claims 1 to 5, or a composition for use according to claim 6 or 7, wherein the neuromuscular disorder is characterized by an impaired neuromuscular transmission and/or an denervation at the NMJ (neuromuscular junction).

9. An anticholinergic compound for use according to any one of claim 1 to 5 or 8, or a composition for use according to any one of claims claims 6 to 8, wherein the neuromuscular disorder is characterized by at least one of:
muscarinic overexcitability,
motor neuron death,
impaired synaptic transmission and
neuromuscular junction (NMJ) denervation.

10. An anticholinergic compound for use according to any one of claims 1 to 5 or 8 to 9, or a composition for use according to any one of claims claims 6 to 9, wherein the neuromuscular disorder is selected from the group consisting of: amyotrophic lateral sclerosis (ALS), spinal muscular atrophy (SMA), myasthenia gravis (MG), congenital myasthenia, Lambert-Eaton myasthenic syndrome (LEMS), Lyme disease, poliomyelitis. post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy.

11. An anticholinergic compound for use according to any one of claims 1 to 5 or 8 to 10, or a composition for use according to any one of claims claims 6 to 10, wherein the compound is administered at disease onset or within 1, 2, 3, 4, 5, 6, or 7 weeks following disease onset.

12. An anticholinergic compound for use according to any one of claims 1 to 5 or 8 to 11, or a composition for use according to any one of claims claims 6 to 11, wherein disease onset includes at least one of the symptoms selected from the group consisting of: muscle twitches, muscle cramps, spasticity, muscle weakness, slurred and/or nasal speech, difficulty chewing or swallowing, dysphagia, dysarthria and dyspnea.

13. An anticholinergic compound for use according to any one of claims 1 to 5 or 8 to 12, or a composition for use according to any one of claims 6 to 12, wherein the neuromuscular disorder is analyzed via electrophysiological assessment; pharmacodynamic assessment; the level of neurofilaments (e.g. neurofilament light chain (NFL)) in blood serum, plasma and/or cerebrospinal fluid (CSF); or NMJ biopsies.

14. An anticholinergic compound for use according to any one of claims 1 to 5 or 8 to 13, or a composition for use according to any one of claims claims 6 to 13, wherein the administration of said anticholinergic compound or composition to said human subject results in one or more of the following therapeutic effects:
an increase of the number or percentage of fully innervated NMJ in the subject, maintenance of the number or percentage of fully innervated NMJ in the subject, the decrease of the number or percentage of fully denervated NMJ in the subject, an improvement of the reliability of synaptic transmission, a prevention, stabilization or reduction of motor neuron death in the subject; and/or
an improvement of the motor performance and/or grip strength of the subject; and/or
an improvement of the contractile properties of a muscle at the NMJ of the subject; and/or
an improvement of the resistance to fatigue of a muscle at the NMJ of the subject; and/or
an induction of an increase of the muscle weight at the NMJ of the subject; and/or
an improvement of the quality of life or the delay in the apparition of the deterioration of the quality of life of the subject; and/or
a reduction of the muscarinic activity (or a reduction of the muscarinic hyperexcitability) of perisynaptic Schwann cells (PSC) in the subject, or of the NMJ repair in the subject.

15. An anticholinergic compound for use according to any one of claims 1 to 5 or 8 to 14, or a composition for use according to any one of claims 6 to 14, wherein said treatment results in a stabilization of said disorder.

16. An anticholinergic compound for use according to any one of claims 1 to 5 or 8 to 15, or a composition for use according to any one of claims 6 to 15, wherein treatment of the neuromuscular disorder results in an improvement, relative to a human subject not being treated with the anticholinergic compound or the composition, in an electrophysiological assessment; in a pharmacodynamic assessment; in neurofilaments in blood serum, plasma and/or cerebrospinal fluid (CSF); or in NMJ biopsies of the treated human subject.
